SEQUENCE LISTING (14 sequences)

SEQ ID NO: 1 - DNA, 281 bp, synthetic construct
  source 1..281: mol_type = other DNA
ggcttgttgtccacaaccgttaaaccttaaaagctttaaaagccttatatattcttttttttcttataaaacttaaaaccttagaggctatttaagttgctgatttatattaattttattgttcaaacatgagagcttagtacgtgaaacatgagagcttagtacgttagccatgagagcttagtacgttagccatgagggtttagttcgttaaacatgagagcttagtacgttaaacatgagagcttagtacgtactatcaacaggttgaactgctgatc

SEQ ID NO: 2 - DNA, 70 bp, synthetic construct
  source 1..70: mol_type = other DNA
ttcgcacatcttgttgtctgattattgatttttggcgaaaccatttgatcatatgacaagatgtgtatct

SEQ ID NO: 3 - DNA, 137 bp, synthetic construct
  source 1..137: mol_type = other DNA
agaattggtaaagagagtcgtgtaaaatatcgagttcgcacatcttgttgtctgattattgatttttggcgaaaccatttgatcatatgacaagatgtgtatctaccttaacttaatgattttgataaaaatcatta

SEQ ID NO: 4 - DNA, 22 bp, synthetic construct
  source 1..22: mol_type = other DNA
  misc_feature 20..22: note = PAM sequence
cagggttctggatatctgtggg

SEQ ID NO: 5 - DNA, 2265 bp, synthetic construct
  source 1..2265: mol_type = other DNA
agcttctgacctcttctcttcctcccacagggcctcgagagatctggcagcggagagggcagaggaagtcttctaacatgcggtgacgtggaggagaatcccggccctaggctcgagatggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccagacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgt

SEQ ID NO: 6 - DNA, 2264 bp, synthetic construct
  source 1..2264: mol_type = other DNA
agcttctgacctcttctcttcctcccacagggcctcgagagatctggcagcggagagggcagaggaagtcttctaacatgcggtgacgtggaggagaatcccggccctaggctcgagatggagtttgggctgagctggctttttcttgtggctattttaaaaggtgtccagtgctctagagatattttgctgacccaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccagacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgt

SEQ ID NO: 7 - DNA, 4974 bp, synthetic construct
  source 1..4974: mol_type = other DNA
agcttctgacctcttctcttcctcccacagggcctcgagagatctggcagcggagagggcagaggaagtcttctaacatgcggtgacgtggaggagaatcccggccctaggctcgagatggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcgtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcagccaaaacgacacccccatcagtctatggaagggtcaccgtctcttcagcggagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgggtccggacagtgcaccaattacgccctcctgaagcttgctggcgacgtggagagcaaccctggtcccatgggggcaggtgccactggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccatgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggtcttgtggcctacatagccttcaagaggtggaacagccgcgccaagcgctcgggttcgggttaaacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgttcgagggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgggctccggggctaccaatttcagcctgctcaagcaggccggagacgtggaggaaaaccctggtcccatgcctgaaccctctaagtctgctccagcccctaaaaagggttctaagaaggctatcactaaggcgcagaagaaggatggtaagaagcgtaagcgcagccgcaaggagagctattctatctatgtgtacaaggttctgaagcaggtccaccccgacaccggcatctcatccaaggccatggggatcatgaactccttcgtcaacgacatcttcgagcgcatcgcgggcgaggcttctcgcctggctcactacaataagcgctcgaccatcacctccagggagattcagacggctgtgcgcctgctgctgcctggggagctggctaagcatgctgtgtccgagggcactaaggcagttaccaagtacactagctctaaggatccaccggtcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataactcgagagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggct

SEQ ID NO: 8 - RNA, 19 nt, synthetic construct
  source 1..19: mol_type = other RNA
cagggttctggatatctgt

SEQ ID NO: 9 - DNA, 22 bp, synthetic construct
  source 1..22: mol_type = other DNA
ggcctttttcccatgcctgcct

SEQ ID NO: 10 - DNA, 22 bp, synthetic construct
  source 1..22: mol_type = other DNA
tgccctctcctgccaccttctc

SEQ ID NO: 11 - DNA, 58 bp, synthetic construct
  source 1..58: mol_type = other DNA
tcctaaccctgatcctcttgtcccacagatatccagaaccctgaccctgccctgtacc

SEQ ID NO: 12 - DNA, 58 bp, synthetic construct
  source 1..58: mol_type = other DNA
  misc_feature 35..37: note = PAM sequence
  misc_feature 16..34: note = Protospacer (P) sequence
ggtacacggcagggtcagggttctggatatctgtgggacaagaggatcagggttagga

SEQ ID NO: 13 - DNA, 22 bp, synthetic construct
  source 1..22: mol_type = other DNA
cccacagatatccagaacccgt

SEQ ID NO: 14 - DNA, 100 bp, synthetic construct
  source 1..100: mol_type = other DNA
  misc_feature 30..32: note = PAM sequence
  misc_feature 33..51: note = Protospacer (P) sequence
agatcatgtcctaaccctgatcctcttgtcccacagatatccagaaccctgaccctgccgtgtaccagctgagagactctaaatccagtgacaagtctgt

(19) United States
(12) Patent Application Publication
(10) Pub. No.: US 2026/0061076 A1
(43) Pub. Date: Mar. 5, 2026

(54) METHODS OF GENOME EDITING OF CELLS WITH MODIFIED DONOR TEMPLATES

(71) Applicant: Wisconsin Alumni Research Foundation, Madison, WI (US)

(72) Inventors: Krishanu Saha, Middleton, WI (US); Anna Tommasi, Madison, WI (US); Madison Bugel, Bethesda, MD (US); Daniel Cappabianca, Madison, WI (US)

(73) Assignee: Wisconsin Alumni Research Foundation, Madison, WI (US)

(21) Appl. No.: 19/316,786

(22) Filed: Sep. 2, 2025

Related U.S. Application Data
(60) Provisional application No. 63/690,158, filed on Sep. 3, 2024.

(51) Int. Cl. A61K 48/00; A61K 35/17; A61K 40/11; A61K 40/31; C07K 14/725; C12N 9/22; C12N 15/11; C12N 15/85; C12N 15/90

(52) U.S. Cl. CPC A61K 48/005; A61K 35/17; A61K 40/11; A61K 40/31; C07K 14/7051; C12N 9/226; C12N 15/111; C12N 15/85; C12N 15/907; A61K 2239/13; A61K 2239/21; A61K 2239/22; A61K 2239/48; C12N 2310/20; C12N 2830/50

(57) ABSTRACT

Described herein is a DNA template plasmid for generating genome modified immune cells including a plasmid backbone, a first insert, and a second insert. The first insert includes a transgene, wherein the transgene is flanked by left and right homology arms that are complementary to sequences on both sides of a cleavage site in a target expressed gene in an unmodified immune cell. The second insert includes a cleavage target including a protospacer sequence defining the cleavage site in the target expressed gene, and a protoadjacent motif sequence (PAM) for recognition by a Cas9 ribonucleoprotein complex (Cas9-RNP). The Cas9-RNP includes a Cas9 polypeptide and a single guide RNA (sgRNA) comprising a sequence complementary to the protospacer sequence. The Cas9-RNP binds the second insert and linearizes the DNA template plasmid by Cas9-RNP-directed cleavage at the cleavage site. Also included are RNP complexes, methods of genome modifying immune cells, and treatment methods.

### CROSS-REFERENCE TO RELATED APPLICATIONS

This application claims priority to U.S. Provisional Application 63/690,158 filed on Sep. 3, 2024, which is incorporated herein by reference in its entirety.

### STATEMENT REGARDING FEDERALLY SPONSORED RESEARCH & DEVELOPMENT

This invention was made with government support under GM119644 awarded by the National Institutes of Health and under EEC1648035 awarded by the National Science Foundation. The government has certain rights in the invention.

### SEQUENCE LISTING

The Instant Application contains a Sequence Listing which has been submitted electronically in XML format and is hereby incorporated by reference in its entirety. Said XML copy, created on Aug. 25, 2025, is named “SEQ_LIST—107668331-P240294US02.xml” and is 25,339 bytes in size. The Sequence Listing does not go beyond the disclosure in the application as filed.

### FIELD OF THE DISCLOSURE

The present disclosure is related to a method and plasmid construct for creating genome edited cells, (e.g., CAR T) cells using CRISPR-Cas gene editing.

### BACKGROUND

Chimeric antigen receptor (CAR) T cells demonstrate the promise of engineering primary T cells to advance human health. However, current techniques to manufacture CAR T cells are limited, making it difficult to scale up manufacturing and keep up with the demand for these therapies. In addition, the use of viral vectors to encode CARs in the genome pose significant risks to patients such as insertional mutagenesis that could lead to gene silencing or oncogene activation. Thus, there is a need to optimize CAR T manufacturing processes and non-virally deliver CAR transgenes precisely to eliminate risks that viral vectors pose.

CRISPR/Cas9 genome editing has emerged as an alternative to viral transduction of T cells. Short guide RNA (gRNA) sequences can complex with Cas9 nuclease to form a ribonucleoprotein (RNP) capable of producing double-strand breaks (DSBs) at a targeted location in the genome. These DSBs are repaired via error-prone non-homologous end joining (NHEJ) to knock genes out or homology-directed repair (HDR) to incorporate exogenous DNA. While the CAR transgene has been incorporated successfully at multiple loci including TRAC in human cells, transgene integration specifically upstream of the endogenous T cell receptor, alpha chain (TRAC) promoter produces TRAC-CAR T cells with a stem cell memory-like phenotype that are more resistant to exhaustion. A more stem-cell like CAR T cell product is correlated to increased persistence in vivo, which is beneficial for targeting hematological and solid tumors.

TRAC-CAR T cell manufacturing historically has relied on linear, double-stranded DNA (dsDNA) HDR donor template generated via polymerase chain reaction (PCR). Another approach is linear single-stranded DNAs (ssDNA) using templates that include a truncated Cas9 target sequence (tCTS), for example. Both of these approaches, however, are inefficient at transgene knock-in, can suffer from imprecise encoding of the transgene, and can be toxic to the cells affecting cell viability. In a further modification, gRNA target sequences have been incorporated into plasmid dsDNA to produce RNP-based linear DNA templates which generate genome modified cells via homology-mediated end-joining (HMEJ) transgene knock-in. However, HMEJ can result in non-targeted insertion of the transgene.

What is needed are alternative methods for transgene insertion to provide genome edited cells which improve targeting, viability and manufacturing scale-up.

### BRIEF SUMMARY

In an aspect, a DNA template plasmid for generating genome modified immune cells comprises a plasmid backbone, a first insert, and a second insert;
wherein the first insert comprises a transgene, wherein the transgene is flanked by left and right homology arms that are complementary to sequences on both sides of a cleavage site in a target expressed gene in an unmodified immune cell;
wherein the second insert comprises a cleavage target comprising a protospacer sequence defining the cleavage site in the target expressed gene, and a protoadjacent motif sequence (PAM) for recognition by a Cas9 ribonucleoprotein complex (Cas9-RNP);
wherein the Cas9-RNP comprises a Cas9 polypeptide and a single guide RNA (sgRNA) comprising a sequence complementary to the protospacer sequence; and
wherein the Cas9-RNP binds the second insert and linearizes the DNA template plasmid by Cas9-RNP-directed cleavage at the cleavage site.

In another aspect, an RNP complex comprises the DNA template plasmid described above, the Cas9 polypeptide, and the sgRNA.

In another aspect, an ex vivo, virus-free method of site-specifically inserting a transgene into an immune cell expressed gene to generate a genome modified immune cell, comprising
providing the DNA template plasmid described above,
incubating the DNA template plasmid with the Cas9-RNP for a time sufficient to linearize the DNA template plasmid by Cas9-RNP-directed cleavage at the cleavage site, and
introducing into a population of unmodified immune cells the Cas9-RNP and linearized DNA template plasmid to provide the genome modified immune cells, wherein, in the genome modified immune cells, the transgene is specifically integrated into the cleavage site of the immune cell expressed gene.

In a further aspect, the method further comprises administering the genome modified immune cell to a subject in need of such treatment.

### BRIEF DESCRIPTION OF THE DRAWINGS

FIGS. 1A-G demonstrate robust manufacturing of TRAC-CAR T cells with Cas9-Cleaved, LInearized with Protein Template (CLIPT). (1A) An SpCas9 RNP complex targeted to exon 1 of the TRAC locus (SEQ ID NOs: 11 and 12) was used to insert dsDNA HDR donor templates containing an anti-GD2 CAR transgene under the control of the endogenous TRAC promoter: Cas9-CLIPT and Control template. Cas9-CLIPT contained a TRAC gRNA site with PAM to be linearized by the RNP with TRAC sgRNA. TRAC sgRNA plus PAM (5′-CAGGGTTCTGGATATCTGTGGG-3′; SEQ ID NO: 4) and complementary sequence (CCCACAGATATCCAGAACCCGT: SEQ ID NO: 13) are shown. The control template was linearized via an Sspl restriction site. (1B) Cas9-CLIPT or Control DNA (+/−PGA) were incubated with RNP for 10 minutes and run on a 1% agarose gel (Lanes 2-5) with a 1 kb ladder (Lanes 1-10) and controls (Lanes 6-9). (1C) T cells were activated with anti CD3/CD28 beads in TexMACS™ for 3 days, electroporated (EP) with RNP and Nanoplasmid™ donor templates, and expanded in ImmunoCult™ XF supplemented with IL-7/IL-15 until Day 10 to manufacture TRAC-CAR T cells. (1D) Representative contour plots of Cas9-CLIPT, Control TRAC-CAR T cells or non-transfected samples analyzed for CAR and TCR expression on Day 8 (Day 5 post-EP). Bar graphs of Cas9-CLIPT or Control TRAC-CAR T cell (1E) CAR or TCR expression on Day 8 and 10 of manufacturing and (1F) fold-expansion and viability on Day 10 with treatment with M3814 and PGA. (1G) CAR and TCR expression of Cas9-CLIPT and Control TRAC-CAR T cells manufactured at small (5e6 cells) and large (50e6 cells) scales on the Cellares electroporation device, without treatment with M3814 or PGA. Cas9-CLIPT, cleaved, linearized with protein template; dsDNA, double-stranded DNA; RNP, ribonucleoprotein; TRAC, T-cell receptor alpha constant; CAR, chimeric antigen receptor; EP, electroporation. 3 donors, N_{Cas9-CLIPT}=N_{Control}=6 (3 donors), N_{Cas9-CLIPT Small CEP}=N_{Cas9-CLIPT Large CEP}=2, N_{Control Small CEP}=1 (3 separate donors); Error bars represent mean and standard deviation. Statistical significance was determined with paired-t-tests; **** p<0.0001.

FIGS. 2A-C show long-read sequencing of on-target TRAC edits in CAR T cells (SEQ ID NO: 14). Genomic DNA was extracted on Day 10 of manufacturing from Cas9-CLIPT CAR T cells, Control TRAC-CAR T cells, and non-transfected T cells. Regions surrounding the cut site were amplified via PCR and amplicons were sequenced on the PromethION™ 24. (2A) Indel editing efficiency in genomic DNA isolated from the CAR T products as measured by long-read sequencing. The modification frequency of each base pair around the TRAC cut site was calculated relative to the human genome reference sequence. The protospacer (P) and PAM are underlined, and a dotted line indicates where Cas9 nuclease should generate a dsDNA DSB. (2B) Alignment of long-reads from sequencing the genomic DNA of Cas9-CLIPT CAR T cell from Donor A. Gray indicates the relative number of reads at each base pair aligned to the insert and homology arms with TRAC above. Light gray is the negative strand, while dark gray is the positive strand. (2C) Allelic knock-in efficiency as measured by long-read sequencing. Efficiency is defined as the number of reads aligned to the insert divided by the total number of reads aligned to both the transgene insert and the reference genome. NCas9-CLIPT=N_{Control}=3. Error bars represent mean and standard deviation. Statistical significance was calculated with paired t-test; * p<0.05.

FIGS. 3A-C show whole genome sequencing of Cas9-CLIPT TRAC-CAR T cells indicates minimal off-target effects. Genomic DNA was extracted from Cas9-CLIPT TRAC-CAR T cells and sequenced via Oxford Nanopore Sequencing for unbiased whole genome sequencing to determine off-target sites. (3A) Schematic of unbiased whole genome sequencing alignment. Sequencing reads were aligned to the transgene and hits were then mapped to the human genome. After filtering with an overlap window threshold, reads that aligned to both the transgene and human genome were considered an off-target integration site. (3B) Percent of reads that were considered an off-target integration site on each chromosome on a log scale. Chromosome 14 contains the on-target TRAC locus for Cas9-CLIPT TRAC-CAR T cells. (3C) The number of integration site reads on each chromosome, and the corresponding average alignment length of the read to the transgene (14g2a) and human genome (chromosomal). The average MAPQ of each alignment is also reported. 1 donor. NCas9-CLIPT=1.

FIGS. 4A-F show Cas9-CLIPT TRAC-CAR T cells are stem cell memory-like and potent against solid tumor cells in vitro. (4A) Cas9-CLIPT TRAC-CAR T cells were analyzed for expression of stem cell memory T cell surface markers by spectral flow cytometry on Day 7 post-EP. (4B) Representative contour plots of CD62L vs CCR7 expression in CD45RA+/− populations of Cas9-CLIPT TRAC-CAR T cells. (4C) Percentage of Naïve (CD45RA⁺/CD62L⁺/CCR7⁺), Naïve-Central Memory (CD45RA⁺/CD62L⁺/CCR7⁻), Central Memory (CD45RA⁻/CD62L⁺/CCR7⁺), Central-Effector Memory (CD45RA⁻/CD62L⁺/CCR7⁻), Effector Memory (CD45RA⁻/CD62L⁺/CCR7⁺), and terminal effector (CD45RA⁺/CD62L⁺/CCR7⁻) T cells. 3 donors. (4D) GD2⁺ neuroblastoma CHLA-20 cells were plated in 96-well plates 24 hrs before TRAC-CAR T cell addition. The potency was measured continuously for up to 72 hours. (4E) Percentage change in GFP fluorescence from GD2⁺ CHLA-20 neuroblastoma cells versus time in cancer/Cas9-CLIPT or Control TRAC-CAR T cell co-cultures for a 1.25:1 E:T ratio. (4F) Percent cytotoxicity at 72 hours for cancer/Cas9-CLIPT and Control TRAC-CAR T cells at an E:T ratio of 1.25:1. GFP, green fluorescent protein; E:T, effector: target ratio; 3 donors, N_{Cas9-CLIPT}=N_{Control}=6. Error bars represent mean and standard deviation. Statistical significance was determined with One-Way ANOVA; * p<0.05; ** p<0.01.

FIGS. 5A-D show large knock-in using Cas9-CLIPT enables fluorescent reporter of CAR activation. (5A) Schematic of Cas9-CLIPT NFAT-mCh DNA containing an anti-GD2 CAR transgene under the control of the endogenous TRAC promoter and an mCherry reporter gene conditionally expressed with NFAT binding domains and the minimal IL-2 promoter was targeted to the TRAC locus. Cas9-CLIPT templates contain a TRAC gRNA site with PAM to be linearized by the RNP (SEQ ID NO. 4 and complementary sequence SEQ ID NO: 13. (5B) NFAT-mCh CAR T cells contain an inducible mCherry reporter gene dependent on CAR activation. A representative contour plot and bar graph depict their CAR and TCR expression. (5C) 1 million NFAT-mCh CAR T cells were stimulated with either PMA/Ionomycin or 200,000 CHLA-20 neuroblastoma cells for 24 hours. Treated and untreated Cas9-CLIPT NFAT-mCh or Cas9-CLIPT TRAC-CAR T cell samples were then assayed for NFAT-driven mCherry expression. Representative contour plots of CAR vs. mCherry expression. (5D) Bar graphs of percentage of CAR⁺/mCherry High populations or mCherry MFI. 3 donors, NCas9-CLIPT=N_{Cas9-CLIPT I-mCh}=N_{Control}=6. Error bars represent mean and standard deviation. Statistical significance was determined with One-Way ANOVA; **** p<0.0001.

FIGS. 6A-E show Long-read sequencing of on-target TRAC edits in potent NFAT-mCh CAR T cells (SEQ ID NO: 14). Genomic DNA was extracted on Day 10 of manufacturing from Cas9-CLIPT, NFAT-mCh, and Control TRAC-CAR T cells as well as non-transfected T cells. Regions surrounding the cut site were amplified via PCR and amplicons were sequenced on the PromethION 24. (6A) Indel editing efficiency in genomic DNA isolated from the CAR T products as measured by long-read sequencing. The modification frequency of each nucleotide around the TRAC cut site was calculated relative to the human genome TRAC reference sequence. The protospacer (P) and PAM are underlined, and a vertical dotted line indicates where Cas9 nuclease should create a dsDNA DSB. (6B) Allelic knock-in efficiency as measured by long-read sequencing. Knock-in efficiency is defined as the number of reads aligned to the transgene divided by the total number of reads aligned to both the transgene and the reference genome. (6C) GD2⁺ neuroblastoma GFP+CHLA-20 cells were plated in 96-well plates 24 hours before TRAC-CAR T cell addition. GFP expression was measured continuously for up to 72 hours. (6D) “(D) Percent change in GFP fluorescence from GD2+ CHLA-20 neuroblastoma cells versus time in cancer/NFAT-mCh CAR T cell co-cultures for E:T ratios of 10:1, 5:1, 2.5:1, or 1.25:1” and (E) bar graph of extent of cytotoxicity at 72 hours for 1.25:1 E:T ratios. Error bars represent mean and standard deviation. 3 donors, N_{Cas9-CLIPT NFAT-mCh}=6. Error bars represent mean and standard deviation. Statistical significance was determined with paired t-tests; * p<0.05; ** p<0.01; **** p<0.0001.

The above-described and other features will be appreciated and understood by those skilled in the art from the following detailed description, drawings, and appended claims.

### DETAILED DESCRIPTION

Described herein is a DNA template plasmid for generating genome modified immune cells (e.g., a Nanoplasmid™ template) referred to herein as Cas9-CLIPT, Cleaved, LInearized with Protein Template, that contains a single Cas9 gRNA target sequence and an HDR transgene template to improve knock-in via HDR. This strategy facilitates in vitro dsDNA linearization thereby eliminating the need for restriction enzyme-based linearization. Importantly, the strategy simplifies the amount of necessary gene editing reagents and thus reduces manufacturing time, manufacturing cost, and human error. The Cas9-CLIPT template exemplified herein encodes an anti-GD2 CAR that can be used to produce TRAC-CAR T cells reaching HDR knock-in efficiencies up to 60%. Improved HDR knock-in was demonstrated on several electroporation instruments at large-scale, relevant for clinical-scale manufacturing. Further, TRAC-CAR T cells with a large transgene insertion (5.5 kb) can be generated using Cas9-CLIPT manufacturing workflows. The Cas9-CLIPT platform can produce high yields of TRAC-CAR T cells that maintain potency against solid tumor targets and stem cell memory phenotype. These advances could streamline non-viral CAR T cell manufacturing to increase cell yield and simplify supply chains.

In an aspect, described herein are genome modified immune cells, DNA template plasmids for preparing genome modified immune cells, and methods of making genome modified immune cells.

In some aspects, the immune cell is a T-cell, a Natural Killer (NK) cell, an innate lymphoid cell, a Cytokine Induced Killer (CIK) cell, a hematopoietic progenitor cell, a peripheral blood (PB) derived immune cell, a bone marrow derived immune cell, a macrophage, or an umbilical cord blood (UCB) derived immune cell. In some aspects, the immune cell is an embryonic or induced pluripotent stem cell (iPSC)-derived immune cell. In some aspects, the immune cells are modified autologous cells isolated from a patient in need of cancer treatment, or modified cells from an allogeneic healthy donor with intent to treat a patient with cancer.

The immune cells may be isolated from subjects, particularly mammalian subjects such as human subjects and companion animals. The immune cells can be obtained from a healthy donor or from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing.

The population of immune cells can be obtained from a subject in need of therapy or suffering from a disease associated with reduced immune cell activity. Thus, the cells will be autologous to the subject in need of therapy. Alternatively, the population of immune cells can be obtained from a donor such as an allogenic healthy donor. The immune cell population can be harvested from PB, cord blood, bone marrow, spleen, or any other organ/tissue in which immune cells reside in said subject or donor. The immune cells can be isolated from a pool of subjects and/or donors, such as from pooled cord blood. The population of immune cells can be derived from iPSCs and/or any other stem cell known in the art. In some aspects, the iPSCs and/or stem cells used to derive the population of immune cells can be obtained from a subject in need of therapy or suffering from a disease associate with reduced immune cell activity, thus these iPSCs and/or stem cells will be autologous to the subject in need of therapy. Alternatively, the iPSCs and/or stem cells can be obtained from a healthy donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells may or may not be human leukocyte antigen (HLA)-compatible. To be rendered subject-compatible, allogeneic cells can be treated to reduce immunogenicity.

### DNA Template Plasmid

Described herein is a DNA template plasmid for generating genome modified immune cells, e.g. T cells, referred to herein as Cas9-CLIPT. In vitro dsDNA linearization is done using a Cas9 ribonucleoprotein (Cas9-RNP). The Cas9 RNP also provides integration of a transgene into the genome of the T cell. In an aspect, the Cas9-CLIPT DNA is linearized without the use of enzyme-based linearization.

In an aspect, a DNA template plasmid for generating genome modified immune cells comprises, consists essentially of, or consists of, a plasmid backbone, a first insert, and a second insert. The first insert comprises a transgene which is flanked by left and right homology arms that are complementary to sequences on both sides of a cleavage site in a target expressed gene in an unmodified immune cell. The second insert comprises a cleavage target comprising a protospacer sequence defining the cleavage site in the target expressed gene, and a protoadjacent motif sequence (PAM) for recognition by a Cas9 ribonucleoprotein complex (Cas9-RNP). The Cas9-RNP comprises a Cas9 polypeptide and a single guide RNA (sgRNA) comprising a sequence complementary to the protospacer sequence. The Cas9-RNP binds the second insert and linearizes the DNA template plasmid by Cas9-RNP-directed cleavage at the cleavage site.

In an aspect, the method utilizes a single Cas9-RNP, that is a single species of gRNA, which associates with a single protospacer sequence.

### Plasmid Backbone

DNA template plasmid comprises a plasmid backbone. While not required, in some embodiments, the plasmid backbone comprises 500 base pairs or less and does not include an antibiotic-resistance gene.

An exemplary plasmid backbone is described in WO2014077866 and WO2014035457. In an aspect, the plasmid backbone comprises a replication origin selected from the group consisting of Pₘᵢₙ minimal pUC origin, R6K replication origin, ColE2-P9 replication origin, and ColE2 related replication origin. An R6K origin preferably has at least 90% sequence identity to the sequence set forth as SEQ ID NO: 1.

ggcttgttgt ccacaaccgt taaaccttaa aagctttaaa
agccttatat attctttttt ttcttataaa acttaaaacc
ttagaggcta tttaagttgc tgatttatat taattttatt
gttcaaacat gagagcttag tacgtgaaac atgagagctt
agtacgttag ccatgagagc ttagtacgtt agccatgagg
gtttagttcg ttaaacatga gagcttagta cgttaaacat
gagagcttag tacgtactat caacaggttg aactgctgat c

In an aspect, the plasmid backbone comprises an RNA-OUT selection marker. An RNA-OUT selectable marker DNA fragment includes E. coli transcription promoter and terminator sequences flanking an RNA-OUT RNA. RNA-OUT (SEQ ID NO: 2), an antisense RNA that hybridizes to, and reduces translation of, the transposon gene expressed downstream of RNA-IN. RNA-IN is an RNA complementary and antisense to RNA-OUT. When RNA-IN is cloned in the untranslated leader of a mRNA, annealing of RNA-IN to RNA-OUT reduces translation of the gene encoded downstream of RNA-IN.

ttcgcacatc ttgttgtctg attattgatt tttggcgaaa
ccatttgatc atatgacaag atgtgtatct

In an aspect, an RNA-OUT selectable marker, utilizing the RNA-OUT promoter and terminator sequences, that is flanked by Dralll and Kpnl restriction enzyme sites (SEQ ID NO:3). The RNA-OUT promoter and terminator sequences flanking the RNA-OUT RNA may be replaced with heterologous promoter and terminator sequences. For example, the RNA-OUT promoter may be substituted with a CpG free promoter known in the art.

agaattggta aagagagtcg tgtaaaatat cgagttcgca
catcttgttg tctgattatt gatttttggc gaaaccattt
gatcatatga caagatgtgt atctacctta acttaatgat
tttgataaaa atcatta

In an aspect, the plasmid backbone is a Nanoplasmid™, commercially available from Aldevron. In another aspect, the plasmid backbone is a GenCircle™ vector commercially available from GenScript.

### First Insert (Transgene Insert)

The first insert comprises a transgene flanked by left and right homology arms (HA) that are complementary to sequences on both sides of a cleavage site in a target expressed gene in an unmodified immune cell.

In an aspect, the first insert comprises a splice acceptor site (SA), encodes a self-cleaving peptide (2A), includes a terminator sequence that defines the end of a transcriptional sequence (polyA), or a combination thereof.

In an aspect, the transgene has the following structure:
(left HA)-(SA)-(2A)-(transgene)-(poly-A)-(right HA),
wherein left HA and the right HA are the homology arms, wherein SA is a splice acceptor site, wherein 2A encodes a self-cleaving peptide, and wherein poly-A is a is a terminator sequence that defines the end of a transcriptional unit.

In an aspect, the Cas9-CLIP templates described herein permit knock-in of large transgene inserts, such as transgene inserts up to 6.6 kb.

### Transgene

A transgene is a synthetic DNA sequence, e.g., a chimeric antigen receptor (CAR) gene, which is introduced into an unmodified immune cell to produce a genome modified immune cell. As used herein, a synthetic DNA sequence is a DNA sequence that is not native to the genome of the immune cell to be modified. An exemplary aspect of a synthetic DNA sequence is a CAR which refers to a recombinant fusion protein that has an antigen-specific extracellular domain coupled to an intracellular domain that directs the cell to perform a specialized function upon binding of an antigen to the extracellular domain.

A CAR comprises a first extracellular domain linked to a first intracellular domain through a first transmembrane domain, wherein the first extracellular domain comprises an antigen recognition domain.

An antigen-specific extracellular domain specifically binds an antigen when, for example, it binds the antigen with an affinity constant or affinity of interaction (KD) between about 0.1 pM to about 10 μM, specifically about 0.1 pM to about 1 μM, more specifically about 0.1 pM to about 100 nM. Methods for determining the affinity of interaction are known in the art. An antigen-specific extracellular domain suitable for use in a CAR may be any antigen-binding polypeptide, one or more scFv, or another antibody-based recognition domain (cAb VHH (camelid antibody variable domains) or humanized versions thereof, IgNAR VH (shark antibody variable domains) and humanized versions thereof, sdAb VH (single domain antibody variable domains) and “camelized” antibody variable domains are suitable for use. In some instances, T cell receptor (TCR) based recognition domains such as single chain TCR may be used as well as ligands for cytokine receptors.

The antigen recognition domain can bind a cell surface antigen found on a normal cell or a dysfunctional cell, such as a tumor-specific antigen. Exemplary cell surface antigens include B cell surface protein biomarkers such as CD19, CD20, CD34, CD38 and CD45R; senescent cell protein biomarkers such as uPAR, p16, p53 and p21; epithelial cell surface markers such as EpCAM; and additional normal cell surface markers such as CD29, CD9, CD166, CD44, Notch3 and CD123. CD19, for example, is a biomarker of both normal and neoplastic B cells.

The extracellular domain of the CAR can comprise an antigen recognition domain that binds a tumor-specific antigen.

The modified immune cells described herein can express an antigen recognition domain that binds a tumor-specific antigen. The antigen can be expressed as a peptide or as an intact protein or portion thereof. The intact protein or a portion thereof can be native or mutagenized. Non-limiting examples of tumor-specific antigens that are CAR targets include wherein the tumor-specific antigen comprises carbonic anhydrase IX (CAIX), carcinoembryonic antigen (CEA), CD5, CD8, CD7, CD10, CD19, CD20, CD22, CD30, CD33, CLL1, CD34, CD38, CD41, CD44, CD49f, CD56, CD74, CD133, CD138, CD123, CD44V6, Claudin-18, B7 homolog 3 protein (B7-H3), fibroblast activation protein (FAP), cancer antigen 19 (CA19), an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), K-light chain, kinase insert domain receptor (KDR), Lewis Y (LeY), L1 cell adhesion molecule (LICAM), melanoma antigen family A, 1 (MAGE-A1), Mucin 16 (MUC16), Mucin 1 (MUC1), Mesothelin (MSLN), PSMA, GPC3, ERBB2, MAGEA3, p53, MART1, GP100, Proteinase 3 (PR1), Tyrosinase, Survivin, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFR, EGFR-VIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, or a combination thereof.

Specific tumor-specific antigens include GD2, HER2, EGFR, mesothelin, Claudin-18.2, PSMA, B7-H3, IL-13Rα2, FAP, CA19, CD19, CD5, MUC1, or a combination thereof.

The use of CARs is not limited to cancer therapy. CAR immune cells can be used in treatments wherein a population of cells can be specifically targeted and eliminated to provide a therapeutic effect. In another aspect, the antigen can include an antigen such as autoimmune and inflammatory disease antigens such as antigens including B-cell and plasma lineage antigens such as BCMA, GPRC5D, CD20, CD22; cardiac disease antigens such as fibroblast-specific antigens and fibroblast activation protein (FAP), and LRRC15; senescence-associated disease ligands such as NKG2D ligands, MICA, MICB, ULBP1-5, glycoprotein non-metastatic melanoma protein B (GPNMB); other disease-associated antigens such as antigens associated with pulmonary aspergillosis; and the like.

The antigen-specific extracellular domain can also include a spacer linking the Vh and VL chains of the scFV, which can be the hinge region of IgG1 and is sufficient for most scFv-based constructs. Flexible linkers include glycine-serine linkers and Whitlow linkers.

The intracellular domain transmits the immune cell activation signal. The intracellular domain can increase immune cell cytokine production and facilitate immune cell replication. The intracellular domain reduces CAR immune cell exhaustion, increases CAR immune cell antitumor activity, and enhances survival of CAR immune cells in patients. Exemplary intracellular domains comprise co-stimulatory domains, including those from CD27, CD28, CD137 or 4-1BB, CD154 or CD40L, CD244 or 2B4, CD278 or ICOS, CD134 or OX40, CD3-ζ, and combinations thereof, and signaling domains (also called cytotoxicity domains), including those from CD16, DAP10, DAP12, CD28, ICOS, CD27, OX40, CD40L, CD3-ζ, and combinations thereof. A costimulatory domain is derived from the intracellular signaling domains of costimulatory proteins that enhance cytokine production, proliferation, cytotoxicity, and/or persistence in vivo.

Typically, the antigen-specific extracellular domain is linked to the intracellular domain of the CAR by a transmembrane domain, e.g., derived from a CD4, CD8, CD-8 alpha, CD8-beta, CD3-epsilon, CD3-beta, CD28, 4-1BB, OC40, PD-1, LAG-3, CH2CH3 or NKG2D, IgG, CD3-3 transmembrane domain, or combinations thereof. The transmembrane domain traverses the cell membrane, anchors the CAR to the NK cell surface, and connects the extracellular domain to the intracellular signaling domain, thus impacting expression of the CAR on the NK cell surface.

CARs may also further comprise one or more spacers. A spacer or hinge connects (i) the antigen-specific extracellular domain to the transmembrane domain, (ii) the transmembrane domain to a costimulatory domain, (iii) a costimulatory domain to the intracellular domain, and/or (iv) the transmembrane domain to the intracellular domain. For example, inclusion of a spacer domain (e.g., IgG1, IgG2, IgG4, CD28, CD8) between the antigen-specific extracellular domain and the transmembrane domain may affect flexibility of the antigen-binding domain and thereby CAR function. Transmembrane domains, costimulatory domains, and spacers are known in the art.

### Homology Arms

As used herein, homology arms (HA) are complementary to sequences on both sides of the cleavage site in the immune cell expressed gene. The homology arms guide insertion of a synthetic DNA sequence into the immune cell expressed gene by endogenous DNA repair of the double-stranded DNA cleavage induced by Cas9-RNP. The homology arms are 50 to 3000 nucleotides in length and are complementary to sequences on either side of the cut site in the immune cell expressed gene to facilitate incorporation of the synthetic DNA sequence into the genome of the immune cell. Small sequence variations (<100 bases) from complementary sequences could be included to enable barcoding or tracking of various cell types or to increase efficiencies of insertion of the synthetic DNA sequence. The homology arms can be complementary to sequences in the TRAC gene, a T cell receptor beta subunit constant gene (TRBC), AAVS1 (i.e., PPP1R12C), TET2, FAS, BID, CTLA4, PDCD1, CBLB, PTPN6, CIITA and B2M genes.

In an aspect, the length of the homology arms influences the efficiency of synthetic DNA sequence integration. In an aspect, the homology arms are 400 to 1000 base pairs, specifically 450 to 750 base pairs long.

In an aspect, when the immune cell is a T cell, the left homology arm includes 383 to 588 bp of the TRAC locus directly upstream of the cleavage site, and the right homology arm includes 391 to 499 bp of the TRAC locus directly downstream of the cleavage site.

### Splice Acceptor

The splice acceptor site (SA) assists in the splicing of the synthetic DNA sequence into the transcript generated from the endogenous T cell expressed gene. The site at the 3′ end of an intron typically contains an SA. Therefore, after homology directed repair, the SA in the integrated sequence before the synthetic CAR gene assists in splicing in the CAR and downstream sequences into the endogenous transcript driven by the T cell expressed gene promoter (e.g., TRAC promoter).

### Self-Cleaving Peptides

A self-cleaving peptide sequence, e.g., T2A, assists in the separation or cleavage of the translated peptide of the protein product encoded by the synthetic DNA sequence from the protein product of the native T cell expressed gene. Exemplary self-cleaving peptides sequences include viral 2A peptides such as a porcine teschovirus-1 (P2A) peptide, a Thosea asigna virus (T2A) peptide, an equine rhinitis A virus (E2A) peptide, or a foot-and-mouth disease virus (F2A) peptide.

An internal ribosome entry site (IRES) is a site that provides initiation of translation from an internal region of the mRNA. An IRES provides co-expression of two proteins from the same mRNA.

### Poly a Terminator

The polyadenylation (polyA) terminator is a sequence-based element that defines the end of a transcriptional unit within the synthetic DNA sequence and initiate the process of releasing the newly synthesized RNA from the transcription machinery. Exemplary polyA terminators are rabbit beta-globin polyA and a bovine growth hormone polyA.

### Second Insert: Cleavage Target

The second insert comprises a cleavage target for a Cas9 ribonucleoprotein complex (Cas9-RNP). Specifically, the cleavage target comprises one protospacer sequence defining the cleavage site in the target expressed gene, and one protoadjacent motif sequence (PAM) for recognition by the (Cas9-RNP).

Genome editing of the immune cells as described herein uses a CRISPR system, or Cas9 ribonucleoprotein. CRISPR refers to the Clustered Regularly Interspaced Short Palindromic Repeats type II system used by bacteria and archaea for adaptive defense. This system enables bacteria and archaea to detect and silence foreign nucleic acids, e.g., from viruses or plasmids, in a sequence-specific manner. In type II systems, guide RNA (gRNA) interacts with Cas9 and directs the nuclease activity of Cas9 to target DNA sequences complementary to those present in the guide RNA. gRNA base pairs with complementary sequences in target DNA. Cas9 nuclease activity then generates a double-stranded break in the target DNA.

CRISPR/Cas9 is a ribonucleoprotein (RNP) complex. CRISPR RNA (crRNA) includes an about 20 base element that is complementary to a genomic DNA sequence as well as additional elements that are complementary to the transactivating RNA (tracrRNA). The tracrRNA hybridizes to the crRNA and binds to the Cas9 protein, to provide an active RNP complex. Thus, in nature, the CRISPR/Cas9 complex contains two RNA species.

Guide RNA, or gRNA, can be in the form of a crRNA/tracrRNA two guide system, or a single guide RNA. The guide RNA is capable of directing Cas9-mediated cleavage of target DNA. A guide RNA thus contains the sequences necessary for Cas9 binding and nuclease activity and a target sequence complementary to a target DNA of interest (protospacer sequence).

As used herein, a guide RNA protospacer binding sequence refers to the nucleotide sequence of a guide RNA that binds to a target genomic DNA sequence and directs Cas9 nuclease activity to a target DNA locus in the genome of the T cell such the TRAC gene, a T cell receptor beta subunit constant gene (TRBC), AAVS1 (i.e., PPP1R12C), TET2, FAS, BID, CTLA4, PDCD1, CBLB, PTPN6, CIITA and B2M genes. In some embodiments, the guide RNA protospacer binding sequence is complementary to the target DNA sequence. “Complementary” or “complementarity” refers to specific base pairing between nucleotides or nucleic acids. Base pairing between a guide RNA and a target region in exon 1 of the TRAC gene, for example, can be via a DNA targeting sequence that is perfectly complementary or substantially complementary to the guide RNA. As described herein, the protospacer sequence of a single guide RNA may be customized, allowing the targeting of Cas9 activity to a target DNA of interest.

Any desired target DNA sequence of interest may be targeted by a guide RNA target sequence. Any length of target sequence that permits CRISPR-Cas9 specific nuclease activity may be used in a guide RNA. In some embodiments, a guide RNA contains a 20 nucleotide protospacer sequence.

In addition to the protospacer sequence, the cleavage target includes a protospacer adjacent motif (PAM) adjacent to the protospacer region which is a sequence recognized by the CRISPR RNP. Without wishing to be bound to theory, it is thought that the only requirement for a target DNA sequence is the presence of a protospacer-adjacent motif (PAM) adjacent to the sequence complementary to the guide RNA target sequence. Different Cas9 complexes are known to have different PAM motifs. For example, Cas9 from Streptococcus pyogenes has a NGG trinucleotide PAM motif; the PAM motif of N. meningitidis Cas9 is NNNNGATT; the PAM motif of S. thermophilus Cas9 is NNAGAAW; and the PAM motif of T. denticola Cas9 is NAAAAC.

A “Cas9” polypeptide is a polypeptide that functions as a nuclease when complexed to a guide RNA, e.g., an sgRNA or modified sgRNA. That is, Cas9 is an RNA-mediated nuclease. The Cas9 (CRISPR-associated 9, also known as Csn1) family of polypeptides, for example, when bound to a crRNA: tracrRNA guide or single guide RNA, are able to cleave target DNA at a sequence complementary to the sgRNA target sequence and adjacent to a PAM motif as described above. Cas9 polypeptides are characteristic of type II CRISPR-Cas systems. The broad term “Cas9” Cas9 polypeptides include natural sequences as well as engineered Cas9 functioning polypeptides. The term “Cas9 polypeptide” also includes the analogous Clustered Regularly Interspaced Short Palindromic Repeats from Prevotella and Francisella 1 or CRISPR/Cpf1 which is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Additional Class I Cas proteins include Cas3, Cas8a, Cas5, Cas8b, Cas8c, Cas 10d, Case 1, Cse 2, Csy 1, Csy 2, Csy 3, GSU0054, Cas 10, Csm 2, Cmr 5, Cas10, Csx11, Csx 10, and Csf 1. Additional Class 2 Cas9 polypeptides include Csn 2, Cas4, C2c1, C2c3 and Cas13a.

For example, the Cas9 may include, a Cas9 from Neisseria meningitidis, Treponema denticola, Streptococcus thermophilus, Streptococcus pyogenes, Staphylococcus aureus, Francisella novicida, or Campylobacter jejuni, or a variant thereof, or a combination thereof. In some embodiments, the variant may preferably increase specificity; for example, SpyFi Cas9 (Aldevron, Fargo, N. Dak.). In an exemplary embodiment, the Cas9 includes Streptococcus pyogenes Cas9 (SpCas9).

The term “Cas9” or “Cas9 nuclease” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). In some embodiments, a Cas9 nuclease has an inactive (e.g., an inactivated) DNA cleavage domain, that is, the Cas9 is a nickase. Other embodiments of Cas9, both DNA cleavage domains are inactivated. This is referred to as catalytically-inactive Cas9, dead Cas9, or dCas9.

Functional Cas9 mutants are described, for example, in US20170081650 and US20170152508, incorporated herein by reference for its disclosure of Cas9 mutants.

As used herein, the term editing refers to a change in the sequence of the genome at a targeted genomic location. Editing can include inducing either a double stranded break or a pair of single stranded breaks in the genome, such as in an immune cell expressed gene. Editing can also include inserting a synthetic DNA sequence into the genome of the immune cell at the site of the break(s).

As used herein, a Cas9-RNP that targets a T cell expressed gene comprises a Cas9 protein and a guide RNA that directs double stranded DNA cleavage of the T cell expressed gene. The guide RNA thus includes a crRNA comprising a single-stranded protospacer sequence and a first complementary strand of a binding region for the Cas9 polypeptide, and a tracrRNA comprising a second complementary strand of the binding region for the Cas9 polypeptide, wherein the crRNA and the tracrRNA hybridize through the first and second complementary strands of the binding region for the Cas9 polypeptide. The single-stranded protospacer region of the guide RNA hybridizes to a sequence in the T cell expressed gene, directing cleavage of the T-cell expressed gene to a specific locus of the T cell expressed gene.

Exemplary T cell expressed genes which can be cleaved by the methods described herein include the AAVS1 (i.e., PPP1R12C), TET2, FAS, BID, CTLA4, PDCD1, CBLB, PTPN6, CIITA, B2M, TRAC and TRBC genes, specifically TRAC. The T cell expressed gene—targeted by Cas9 ribonucleoprotein—may result in a reduction or elimination of expression of functional TRAC gene product (e.g., knockout of expression of functional TRAC gene product).

Also included herein is an RNP complex comprising the DNA template plasmid of any of the foregoing claims, the Cas9 polypeptide, and the sgRNA.

### Methods of Gene Editing

Also included herein is an ex vivo, virus-free method of site-specifically inserting a transgene into an immune cell expressed gene to generate a genome modified immune cell, comprising
providing the DNA template plasmid described herein;
incubating the DNA template plasmid with the Cas9-RNP for a time sufficient to linearize the DNA template plasmid by Cas9-RNP-directed cleavage at the cleavage site; and
introducing into a population of unmodified immune cells the Cas9-RNP and linearized DNA template plasmid to provide the genome modified immune cells, wherein, in the genome modified immune cells, the transgene is specifically integrated into the cleavage site of the immune cell expressed gene.

As used herein, “introducing” means refers to the translocation of the Cas9 RNP and DNA template from outside a cell to inside the cell, such as inside the nucleus of the cell. Introducing can include transfection, electroporation, contact with nanowires or nanotubes, receptor mediated internalization, translocation via cell penetrating peptides, liposome mediated translocation, transduction with putative non-integrating viruses (e.g., adeno-associated virus, AAV), viral-like particles (VLPs), and the like.

In an aspect, introducing the Cas9-RNP and linearized DNA template plasmid comprises introducing Cas9-RNP bound to the linearized DNA template plasmid and unbound Cas9-RNP.

The genome modified immune cells may then be cultured, for example in xeno-free medium to provide a cultured population of genome modified immune cells having the transgene sequence specifically integrated in the immune cell expressed gene locus. The term “xeno” comes from the Greek “xenos” meaning strange. Xeno-free (or xenogeneic-free) therefore means free from “strange” components, or components from a “strange” species (strange being relative to the native species you're working with). In terms of cell culture, this would mean human cell lines can be cultured using human-derived components (like human serum), and it is considered xeno-free, since there is no difference between species.

As used herein culturing the genome modified immune cells can include recovery from integration of the transgene sequence and/or expansion of the edited T cell population.

### Methods of Treating and Pharmaceutical Compositions

In an aspect, the modified immune cells described herein are particularly useful to treat solid tumors such as a sarcoma, adrenocortical carcinoma, retinoblastoma, kidney cancer, bladder cancer, breast cancer, neuroblastoma, melanoma, sarcoma, neuroendocrine cancer, colorectal cancer, lung cancer, head and neck cancer, prostate cancer, pancreatic cancer, ovarian cancer, uterine cancer, oral cavity cancer, glioblastoma, lymphoma, diffuse midline glioma, carcinoid tumors, neuroendocrine tumors, thyroid cancer, liver cancer, or a combination thereof. Solid tumors can be primary or metastatic solid tumors. In an aspect, the solid tumor is a poorly immunogenic solid tumor.

Modified immune cell therapy can also be used to treat hematologic malignancies like acute lymphoblastic leukemia, non-Hodgkin large B-cell lymphomas, chronic lymphocytic leukemia and multiple myeloma.

In an aspect, the modified immune cells produced by the methods described herein have activity against a neurodegenerative disease, stroke, craniocerebral trauma and/or accident, or an elderly patient in need of treatment for aging, for example. Thus, the methods further comprise administering the cultured population of modified immune cells to a patient in need of treatment for a neurodegenerative disease, stroke, craniocerebral trauma and/or accident, or an elderly patient in need of treatment for aging. Exemplary neurodegenerative diseases include Alzheimer's disease, dementia, Parkinson's disease, Lewy body disease, ataxia, Huntington's disease, amyotrophic lateral sclerosis, Down syndrome, and spinal muscular atrophy.

“Pharmaceutically acceptable” as used herein means that the compound or composition or carrier is suitable for administration to a subject to achieve the treatments described herein, without unduly deleterious side effects in light of the necessity of the treatment.

The term “effective amount,” as used herein, refers to the amount of the compounds or dosages that will elicit the biological or medical response of a subject, tissue or cell that is being sought by the researcher, veterinarian, medical doctor or other clinician.

The term, “pharmaceutically-acceptable carrier” includes any and all dry powder, solvents, dispersion media, coatings, antibacterial and antifungal agents, isotonic agents, absorption delaying agents, and the like. Pharmaceutically-acceptable carriers are materials, useful for the purpose of administering the compounds in the method of the present invention, which are preferably non-toxic, and may be solid, liquid, or gaseous materials, which are otherwise inert and pharmaceutically acceptable, and are compatible with the compounds described herein. Examples of such carriers include, without limitation, various lactose, mannitol, oils such as corn oil, buffers such as PBS, saline, polyethylene glycol, glycerin, polypropylene glycol, dimethylsulfoxide, an amide such as dimethylacetamide, a protein such as albumin, and a detergent such as Tween 80, mono- and oligopolysaccharides such as glucose, lactose, cyclodextrins and starch.

The term “administering” or “administration,” as used herein, refers to providing the compound or pharmaceutical composition of the invention to a subject suffering from or at risk of the diseases or conditions to be treated or prevented.

A route of administration in pharmacology is the path by which a drug is taken into the body. Routes of administration may be generally classified by the location at which the substance is applied. Common examples may include oral and intravenous administration. Routes can also be classified based on where the target of action is. Action may be topical (local), enteral (system-wide effect, but delivered through the gastrointestinal tract), or parenteral (systemic action, but delivered by routes other than the GI tract), via lung by inhalation. One form of local administration is intratumoral (IT), whereby an agent is injected directly into, or adjacent to, a known tumor site.

A topical administration emphasizes local effect, and substance is applied directly where its action is desired. Sometimes, however, the term topical may be defined as applied to a localized area of the body or to the surface of a body part, without necessarily involving target effect of the substance, making the classification rather a variant of the classification based on application location. In an enteral administration, the desired effect is systemic (non-local), substance is given via the digestive tract. In a parenteral administration, the desired effect is systemic, and substance is given by routes other than the digestive tract.

Examples of parenteral administrations may include intravenous (into a vein), e.g. many drugs, total parenteral nutrition intra-arterial (into an artery), e.g., vasodilator drugs in the treatment of vasospasm and thrombolytic drugs for treatment of embolism, intraosseous infusion (into the bone marrow), intra-muscular, intracerebral (into the brain parenchyma), intracerebroventricular (into cerebral ventricular system), intrathecal (an injection into the spinal canal), and subcutaneous (under the skin). Among them, intraosseous infusion is, in effect, an indirect intravenous access because the bone marrow drains directly into the venous system. Intraosseous infusion may be occasionally used for drugs and fluids in emergency medicine and pediatrics when intravenous access is difficult.

The invention is further illustrated by the following non-limiting examples.

### EXAMPLES

### Materials and Methods

Cell Lines: GD2⁺ human neuroblastoma CHLA-20 cells were gifted by Dr. Mario Otto (University of Wisconsin-Madison). These cells were cultured in Dulbecco's Modified Eagle Medium (DMEM) supplemented with 10% fetal bovine serum (FBS) (Gibco, Thermo Fisher Scientific, Waltham, MA) and 1% penicillin-streptomycin (P/S) (Gibco, Thermo Fisher Scientific, Waltham, MA). AkaLucGFP CHLA-20 cells were created through viral transduction by Dr. James Thomson (Morgridge Institute for Research). In short, Phoenix cells (ATCC, Manassas, VA) were grown in DMEM with 10% FBS and 1% P/S and selected with 1 g/mL of diphtheria toxin (Cayman Biologics, Ann Arbor, MI) and 300 μg/mL hygromycin (ThermoFisher Scientific, Waltham, MA). The selection of transgene-positive cells was confirmed by flow cytometry for mouse Lyt2 expression as a reporter gene (Biolegend, San Diego, CA). 3T3 cells were grown in DMEM with 10% FBS and 1% P/S. Cell authentication was performed using short tandem repeat analysis (Idexx BioAnalytics, Westbrook, Maine, USA) and per ATCC guidelines using cell morphology, growth curves, and Mycoplasma testing within 6 months using the MycoStrip™ Mycoplasma Detection Kit (Invitrogen, Waltham, MA). Cell lines were maintained in culture at 37° C. in 5% CO₂.

Nanoplasmid™ Constructs: A GD2-CAR plasmid construct encoding a 2A.14G2A-CD28-OX40-CD35 CAR gifted by Malcolm Brenner (Baylor College of Medicine) was synthesized and the sequence verified (Control) (GenScript, Piscataway, NJ). An additional sequence encoding the TRAC sgRNA plus PAM (5′-CAGGGTTCTGGATATCTGTGGG-3′; SEQ ID NO: 4) was cloned into the Nanoplasmid™ before the 5′ end of the left homology arm via mutagenesis, synthesized, and the sequence verified (Cas9-CLIPT) (Genscript, Piscataway, NJ). Two other sequences with the TRAC sgRNA were designed in benchling to contain additional transcripts encoding 6 NFAT response elements with minimal IL-2 promoter to drive expression of an H2B-mCherry reporter gene (Cas9-CLIPT NFAT-mCh) that was synthesized and the sequence verified (GenScript, Piscataway, NJ). All transgenes are flanked by 500 bp homology arms and were cloned into a pUC57 backbone. Plasmid products were shipped to Aldevron (Fargo, ND), where they were cloned into a Nanoplasmid™ backbone. Nanoplasmid™ backbones include two components: a small (about 300 bp) R6K origin of replication and an RNA-OUT cassette (about 70 bp). The latter encodes an antisense RNA that enables an antibiotic-free selection of E. coli cells in the presence of sucrose by preventing the expression of genome-integrated levansucrase gene sacB and production of the toxic oligosaccharides from sucrose. In addition, having minimal bacterial backbone leads to a drastic reduction of transgene silencing after genomic insertion. These were manufactured on-site at Aldevron and resuspended in DNase free water at 2 mg/mL. The sequences for the transgenes within the dsDNA donor templates are shown below.

Anti-GD-2 CAR sequence, control:
(SEQ ID NO: 5)
AGCTTCTGACCTCTTCTCTTCCTCCCACAGGGCCTCGAGAGATCTGGCAGCGGA
GAGGGCAGAGGAAGTCTTCTAACATGCGGTGACGTGGAGGAGAATCCCGGCCC
TAGGCTCGAGATGGAGTTTGGGCTGAGCTGGCTTTTTCTTGTGGCTATTTTAAA
AGGTGTCCAGTGCTCTAGAGATATTTTGCTGACCCAAACTCCACTCTCCCTGCC
TGTCAGTCTTGGAGATCAAGCCTCCATCTCTTGCAGATCTAGTCAGAGTCTTGT
ACACCGTAATGGAAACACCTATTTACATTGGTACCTGCAGAAGCCAGGCCAGT
CTCCAAAGCTCCTGATTCACAAAGTTTCCAACCGATTTTCTGGGGTCCCAGACA
GGTTCAGTGGCAGTGGATCAGGGACAGATTTCACACTCAAGATCAGCAGAGTG
GAGGCTGAGGATCTGGGAGTTTATTTCTGTTCTCAAAGTACACATGTTCCTCCG
CTCACGTTCGGTGCTGGGACCAAGCTGGAGCTGAAACGGGCTGATGCTGCACC
AACTGTATCCATCTTCCCAGGCTCGGGCGGTGGTGGGTCGGGTGGCGAGGTGA
AGCTTCAGCAGTCTGGACCTAGCCTGGTGGAGCCTGGCGCTTCAGTGATGATAT
CCTGCAAGGCTTCTGGTTCCTCATTCACTGGCTACAACATGAACTGGGTGAGGC
AGAACATTGGAAAGAGCCTTGAATGGATTGGAGCTATTGATCCTTACTATGGTG
GAACTAGCTACAACCAGAAGTTCAAGGGCAGGGCCACATTGACTGTAGACAAA
TCGTCCAGCACAGCCTACATGCACCTCAAGAGCCTGACATCTGAGGACTCTGCA
GTCTATTACTGTGTAAGCGGAATGGAGTACTGGGGTCAAGGAACCTCAGTCAC
CGTCTCCTCAGCCAAAACGACACCCCCATCAGTCTATGGAAGGGTCACCGTCTC
TTCAGCGGAGCCCAAATCTTGTGACAAAACTCACACATGCCCACCGTGCCCGG
ATCCCAAATTTTGGGTGCTGGTGGTGGTTGGTGGAGTCCTGGCTTGCTATAGCT
TGCTAGTAACAGTGGCCTTTATTATTTTCTGGGTGAGGAGTAAGAGGAGCAGGC
TCCTGCACAGTGACTACATGAACATGACTCCCCGCCGCCCCGGGCCCACCCGCA
AGCATTACCAGCCCTATGCCCCACCACGCGACTTCGCAGCCTATCGCTCCAGGG
ACCAGAGGCTGCCCCCCGATGCCCACAAGCCCCCTGGGGGAGGCAGTTTCCGG
ACCCCCATCCAAGAGGAGCAGGCCGACGCCCACTCCACCCTGGCCAAGATCAG
AGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGC
AGGCCCTGCCCCCTCGCTAACAGCCAGACGCGTGAATTCACTCCTCAGGTGCAG
GCTGCCTATCAGAAGGTGGTGGCTGGTGTGGCCAATGCCCTGGCTCACAAATA
CCACTGAGATCTTTTTCCCTCTGCCAAAAATTATGGGGACATCATGAAGCCCCT
TGAGCATCTGACTTCTGGCTAATAAAGGAAATTTATTTTCATTGCAATAGTGTG
TTGGAATTTTTTGTGTCTCTCACTCGGAAGGACATATGGGAGGGCAAATCATTT
AAAACATCAGAATGAGTATTTGGTTTAGAGTTTGGCAACATATGCCCATATGCT
GGCTGCCATGAACAAAGGTTGGCTATAAAGAGGTCATCAGTATATGAAACAGC
CCCCTGCTGTCCATTCCTTATTCCATAGAAAAGCCTTGACTTGAGGTTAGATTTT
TTTTATATTTTGTTTTGTGTTATTTTTTTCTTTAACATCCCTAAAATTTTCCTTAC
ATGTTTTACTAGCCAGATTTTTCCTCCTCTCCTGACTACTCCCAGTCATAGCTGT
CCCTCTTCTCTTATGGAGATCCCTCGACCTGCAGCCCAAGCTTGGCGTAATCAT
GGTCATAGCTGT
Anti-GD-2 CAR Cas-9 CLIPT:
(SEQ ID NO: 6)
AGCTTCTGACCTCTTCTCTTCCTCCCACAGGGCCTCGAGAGATCTGGCAGCGGA
GAGGGCAGAGGAAGTCTTCTAACATGCGGTGACGTGGAGGAGAATCCCGGCCC
TAGGCTCGAGATGGAGTTTGGGCTGAGCTGGCTTTTTCTTGTGGCTATTTTAAA
AGGTGTCCAGTGCTCTAGAGATATTTTGCTGACCCAACTCCACTCTCCCTGCCT
GTCAGTCTTGGAGATCAAGCCTCCATCTCTTGCAGATCTAGTCAGAGTCTTGTA
CACCGTAATGGAAACACCTATTTACATTGGTACCTGCAGAAGCCAGGCCAGTCT
CCAAAGCTCCTGATTCACAAAGTTTCCAACCGATTTTCTGGGGTCCCAGACAGG
TTCAGTGGCAGTGGATCAGGGACAGATTTCACACTCAAGATCAGCAGAGTGGA
GGCTGAGGATCTGGGAGTTTATTTCTGTTCTCAAAGTACACATGTTCCTCCGCT
CACGTTCGGTGCTGGGACCAAGCTGGAGCTGAAACGGGCTGATGCTGCACCAA
CTGTATCCATCTTCCCAGGCTCGGGCGGTGGTGGGTCGGGTGGCGAGGTGAAG
CTTCAGCAGTCTGGACCTAGCCTGGTGGAGCCTGGCGCTTCAGTGATGATATCC
TGCAAGGCTTCTGGTTCCTCATTCACTGGCTACAACATGAACTGGGTGAGGCAG
AACATTGGAAAGAGCCTTGAATGGATTGGAGCTATTGATCCTTACTATGGTGGA
ACTAGCTACAACCAGAAGTTCAAGGGCAGGGCCACATTGACTGTAGACAAATC
GTCCAGCACAGCCTACATGCACCTCAAGAGCCTGACATCTGAGGACTCTGCAG
TCTATTACTGTGTAAGCGGAATGGAGTACTGGGGTCAAGGAACCTCAGTCACC
GTCTCCTCAGCCAAAACGACACCCCCATCAGTCTATGGAAGGGTCACCGTCTCT
TCAGCGGAGCCCAAATCTTGTGACAAAACTCACACATGCCCACCGTGCCCGGA
TCCCAAATTTTGGGTGCTGGTGGTGGTTGGTGGAGTCCTGGCTTGCTATAGCTT
GCTAGTAACAGTGGCCTTTATTATTTTCTGGGTGAGGAGTAAGAGGAGCAGGCT
CCTGCACAGTGACTACATGAACATGACTCCCCGCCGCCCCGGGCCCACCCGCA
AGCATTACCAGCCCTATGCCCCACCACGCGACTTCGCAGCCTATCGCTCCAGGG
ACCAGAGGCTGCCCCCCGATGCCCACAAGCCCCCTGGGGGAGGCAGTTTCCGG
ACCCCCATCCAAGAGGAGCAGGCCGACGCCCACTCCACCCTGGCCAAGATCAG
AGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGC
AGGCCCTGCCCCCTCGCTAACAGCCAGACGCGTGAATTCACTCCTCAGGTGCAG
GCTGCCTATCAGAAGGTGGTGGCTGGTGTGGCCAATGCCCTGGCTCACAAATA
CCACTGAGATCTTTTTCCCTCTGCCAAAAATTATGGGGACATCATGAAGCCCCT
TGAGCATCTGACTTCTGGCTAATAAAGGAAATTTATTTTCATTGCAATAGTGTG
TTGGAATTTTTTGTGTCTCTCACTCGGAAGGACATATGGGAGGGCAAATCATTT
AAAACATCAGAATGAGTATTTGGTTTAGAGTTTGGCAACATATGCCCATATGCT
GGCTGCCATGAACAAAGGTTGGCTATAAAGAGGTCATCAGTATATGAAACAGC
CCCCTGCTGTCCATTCCTTATTCCATAGAAAAGCCTTGACTTGAGGTTAGATTTT
TTTTATATTTTGTTTTGTGTTATTTTTTTCTTTAACATCCCTAAAATTTTCCTTAC
ATGTTTTACTAGCCAGATTTTTCCTCCTCTCCTGACTACTCCCAGTCATAGCTGT
CCCTCTTCTCTTATGGAGATCCCTCGACCTGCAGCCCAAGCTTGGCGTAATCAT
GGTCATAGCTGT
Anti-GD-2 CAR tNGFR, mCherry:
(SEQ ID NO: 7)
AGCTTCTGACCTCTTCTCTTCCTCCCACAGGGCCTCGAGAGATCTGGCAGCGGA
GAGGGCAGAGGAAGTCTTCTAACATGCGGTGACGTGGAGGAGAATCCCGGCCC
TAGGCTCGAGATGGAGTTTGGGCTGAGCTGGCTTTTTCTTGTGGCTATTTTAAA
AGGTGTCCAGTGCTCTAGAGATATTTTGCTGACCCAAACTCCACTCTCCCTGCC
TGTCAGTCTTGGAGATCAAGCCTCCATCTCTTGCAGATCTAGTCAGAGTCTTGT
ACACCGTAATGGAAACACCTATTTACATTGGTACCTGCAGAAGCCAGGCCAGT
CTCCAAAGCTCCTGATTCACAAAGTTTCCAACCGATTTTCTGGGGTCCCAGACA
GGTTCAGTGGCAGTGGATCAGGGACAGATTTCACACTCAAGATCAGCAGAGTG
GAGGCTGAGGATCTGGGAGTTTATTTCTGTTCTCAAAGTACACATGTTCCTCCG
CTCACGTTCGTGCTGGGACCAAGCTGGAGCTGAAACGGGCTGATGCTGCACCA
ACTGTATCCATCTTCCCAGGCTCGGGCGGTGGTGGGTCGGGTGGCGAGGTGAA
GCTTCAGCAGTCTGGACCTAGCCTGGTGGAGCCTGGCGCTTCAGTGATGATATC
CTGCAAGGCTTCTGGTTCCTCATTCACTGGCTACAACATGAACTGGGTGAGGCA
GAACATTGGAAAGAGCCTTGAATGGATTGGAGCTATTGATCCTTACTATGGTGG
AACTAGCTACAACCAGAAGTTCAAGGGCAGGGCCACATTGACTGTAGACAAAT
CGTCCAGCACAGCCTACATGCACCTCAAGAGCCTGACATCTGAGGACTCTGCA
GTCTATTACTGTGTAAGCGGAATGGAGTACTGGGGTCAAGGAACCTCAGTCAC
CGTCTCCTCAGCCAAAACGACACCCCCATCAGTCTATGGAAGGGTCACCGTCTC
TTCAGCGGAGCCCAAATCTTGTGACAAAACTCACACATGCCCACCGTGCCCGG
ATCCCAAATTTTGGGTGCTGGTGGTGGTTGGTGGAGTCCTGGCTTGCTATAGCT
TGCTAGTAACAGTGGCCTTTATTATTTTCTGGGTGAGGAGTAAGAGGAGCAGGC
TCCTGCACAGTGACTACATGAACATGACTCCCCGCCGCCCCGGGCCCACCCGCA
AGCATTACCAGCCCTATGCCCCACCACGCGACTTCGCAGCCTATCGCTCCAGGG
ACCAGAGGCTGCCCCCCGATGCCCACAAGCCCCCTGGGGGAGGCAGTTTCCGG
ACCCCCATCCAAGAGGAGCAGGCCGACGCCCACTCCACCCTGGCCAAGATCAG
AGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGAC
AAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCC
TTTACCAGGGTCTCAGTACAGCCACCAAGGACACCTACGACGCCCTTCACATGC
AGGCCCTGCCCCCTCGCGGGTCCGGACAGTGCACCAATTACGCCCTCCTGAAGC
TTGCTGGCGACGTGGAGAGCAACCCTGGTCCCATGGGGGCAGGTGCCACTGGC
CGCGCCATGGACGGGCCGCGCCTGCTGCTGTTGCTGCTTCTGGGGGTGTCCCTT
GGAGGTGCCAAGGAGGCATGCCCCACAGGCCTGTACACACACAGCGGTGAGTG
CTGCAAAGCCTGCAACCTGGGCGAGGGTGTGGCCCAGCCTTGTGGAGCCAACC
AGACCGTGTGTGAGCCCTGCCTGGACAGCGTGACGTTCTCCGACGTGGTGAGC
GCGACCGAGCCGTGCAAGCCGTGCACCGAGTGCGTGGGGCTCCAGAGCATGTC
GGCGCCATGCGTGGAGGCCGACGACGCCGTGTGCCGCTGCGCCTACGGCTACT
ACCAGGATGAGACGACTGGGCGCTGCGAGGCGTGCCGCGTGTGCGAGGCGGGC
TCGGGCCTCGTGTTCTCCTGCCAGGACAAGCAGAACACCGTGTGCGAGGAGTG
CCCCGACGGCACGTATTCCGACGAGGCCAACCACGTGGACCCGTGCCTGCCCT
GCACCGTGTGCGAGGACACCGAGCGCCAGCTCCGCGAGTGCACACGCTGGGCC
GACGCCGAGTGCGAGGAGATCCCTGGCCGTTGGATTACACGGTCCACACCCCC
AGAGGGCTCGGACAGCACAGCCCCCAGCACCCAGGAGCCTGAGGCACCTCCAG
AACAAGACCTCATAGCCAGCACGGTGGCAGGTGTGGTGACCACAGTGATGGGC
AGCTCCCAGCCCGTGGTGACCCGAGGCACCACCGACAACCTCATCCCTGTCTAT
TGCTCCATCCTGGCTGCTGTGGTTGTGGGTCTTGTGGCCTACATAGCCTTCAAG
AGGTGGAACAGCCGCGCCAAGCGCTCGGGTTCGGGTTAAACGCGTGAATTCAC
TCCTCAGGTGCAGGCTGCCTATCAGAAGGTGGTGGCTGGTGTGGCCAATGCCCT
GGCTCACAAATACCACTGAGATCTTTTTCCCTCTGCCAAAAATTATGGGGACAT
CATGAAGCCCCTTGAGCATCTGACTTCTGGCTAATAAAGGAAATTTATTTTCAT
TGCAATAGTGTGTTGGAATTTTTTGTGTCTCTCACTCGGAAGGACATATGGGAG
GGCAAATCATTTAAAACATCAGAATGAGTATTTGGTTTAGAGTTTGGCAACATA
TGCCCATATGCTGGCTGCCATGAACAAAGGTTGGCTATAAAGAGGTCATCAGT
ATATGAAACAGCCCCCTGCTGTCCATTCCTTATTCCATAGAAAAGCCTTGACTT
GAGGTTAGATTTTTTTTATATTTTGTTTTGTGTTATTTTTTTCTTTAACATCCCTA
AAATTTTCCTTACATGTTTTACTAGCCAGATTTTTCCTCCTCTCCTGACTACTCC
CAGTCATAGCTGTCCCTCTTCTCTTATGGAGATCCCTCGACCTGCAGCCCAAGC
TTGGCGTAATCATGGTCATAGCTGTTCGAGGGTATCGATAAGCTTGATATCGAA
TTAGGAGGAAAAACTGTTTCATACAGAAGGCGTCAATTAGGAGGAAAAACTGT
TTCATACAGAAGGCGTCAATTAGGAGGAAAAACTGTTTCATACAGAAGGCGTC
AATTGGTCCCATCGAATTAGGAGGAAAAACTGTTTCATACAGAAGGCGTCAAT
TAGGAGGAAAAACTGTTTCATACAGAAGGCGTCAATTAGGAGGAAAAACTGTT
TCATACAGAAGGCGTCAATTGGTCCCGGGACATTTTGACACCCCCATAATATTT
TTCCAGAATTAACAGTATAAATTGCATCTCTTGTTCAAGAGTTCCCTATCACTCT
CTTTAATCACTACTCACAGTAACCTCAACTCCTGGGCTCCGGGGCTACCAATTT
CAGCCTGCTCAAGCAGGCCGGAGACGTGGAGGAAAACCCTGGTCCCATGCCTG
AACCCTCTAAGTCTGCTCCAGCCCCTAAAAAGGGTTCTAAGAAGGCTATCACTA
AGGCGCAGAAGAAGGATGGTAAGAAGCGTAAGCGCAGCCGCAAGGAGAGCTA
TTCTATCTATGTGTACAAGGTTCTGAAGCAGGTCCACCCCGACACCGGCATCTC
ATCCAAGGCCATGGGGATCATGAACTCCTTCGTCAACGACATCTTCGAGCGCAT
CGCGGGCGAGGCTTCTCGCCTGGCTCACTACAATAAGCGCTCGACCATCACCTC
CAGGGAGATTCAGACGGCTGTGCGCCTGCTGCTGCCTGGGGAGCTGGCTAAGC
ATGCTGTGTCCGAGGGCACTAAGGCAGTTACCAAGTACACTAGCTCTAAGGAT
CCACCGGTCGCCACCATGGTGAGCAAGGGCGAGGAGGATAACATGGCCATCAT
CAAGGAGTTCATGCGCTTCAAGGTGCACATGGAGGGCTCCGTGAACGGCCACG
AGTTCGAGATCGAGGGCGAGGGCGAGGGCCGCCCCTACGAGGGCACCCAGAC
CGCCAAGCTGAAGGTGACCAAGGGTGGCCCCCTGCCCTTCGCCTGGGACATCC
TGTCCCCTCAGTTCATGTACGGCTCCAAGGCCTACGTGAAGCACCCCGCCGACA
TCCCCGACTACTTGAAGCTGTCCTTCCCCGAGGGCTTCAAGTGGGAGCGCGTGA
TGAACTTCGAGGACGGCGGCGTGGTGACCGTGACCCAGGACTCCTCCCTGCAG
GACGGCGAGTTCATCTACAAGGTGAAGCTGCGCGGCACCAACTTCCCCTCCGA
CGGCCCCGTAATGCAGAAGAAGACCATGGGCTGGGAGGCCTCCTCCGAGCGGA
TGTACCCCGAGGACGGCGCCCTGAAGGGCGAGATCAAGCAGAGGCTGAAGCTG
AAGGACGGCGGCCACTACGACGCTGAGGTCAAGACCACCTACAAGGCCAAGA
AGCCCGTGCAGCTGCCCGGCGCCTACAACGTCAACATCAAGTTGGACATCACC
TCCCACAACGAGGACTACACCATCGTGGAACAGTACGAACGCGCCGAGGGCCG
CCACTCCACCGGCGGCATGGACGAGCTGTACAAGTAATAACTCGAGAGCCTCG
ACTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTG
CATCGCATTGTCTGAGTAGGTGTCATTCTATTCTGGGGGGTGGGGTGGGGCAGG
ACAGCAAGGGGGAGGATTGGGAAGACAATAGCAGGCATGCTGGGGATGCGGT
GGGCT

Nanoplasmid™ Linearization by Restriction Digest: To linearize Control Nanoplasmid™ for use as a dsDNA HDR template, a restriction digest of the Nanoplasmid™ construct using Sspl-HF (Cat #R3132S, New England Biolabs, Ipswich, MA) was performed. Four restriction digest batch reactions in 1.5 mL Eppendorf Tubes® (50 μL Nanoplasmid™, 125 μL CutSmart® buffer, 25 μL Sspl-HF enzyme, and 1050 μL DNase-free water for 1250 μL total) were aliquoted (50 L into PCR tubes for a total of 96 reactions). These were incubated in a thermocycler at 37° C. for 15-60 min and heat inactivated at 65° C. for 20 min according to the manufacturer's instructions. Gel electrophoresis was then performed on the finished product to assess if proper cutting took place, followed by SPRI cleanups to purify and concentrate the material to 2 mg/mL. PCR reactions were pooled into eight 1.5 mL Eppendorf tubes (600 μL) with an equal volume of SPRI (Beckman-Coulter, Brea, CA) beads that incubated for 5 minutes at room temperature. The product was washed twice with 70% ethanol and eluted in 75 μL of DNase-free water and pooled into one tube (600 μL). This product was subject to a second round of cleanups and was eluted in 30 μL of water. DNA was quantified using the NanoDrop™ 2000 and Qubit™ double-strand DNA (dsDNA) Broad Range (BR) Assay (ThermoFisher Scientific, Waltham, MA) and diluted to 2 mg/mL according to Qubit™ measurements.

PCR-generated dsDNA HDR Template Production: Plasmids were used to make dsDNA PCR-generated donor template with tCTS adapters. In brief, plasmids were MidiPrepped using the Pure Yield™ MidiPrep system (Promega). PCR amplicons were generated from plasmid templates using Q5® Hot Start Polymerase (NEB) and pooled into 100 μL reactions for Solid Phase Reversible Immobilization (SPRI) cleanup (1×) using AMPure XP beads according to the manufacturer's instructions (Beckman Coulter). Each 100 μL starting product was eluted into 5 μL of water. Bead incubation and separation times were increased to 5 min, and elution time was increased to 15 min at 37° C. to improve yield. PCR products from round 1 cleanup were pooled and subjected to a second round of SPRI cleanup (1×) to increase total concentration. Round 2 elution volume was 20% of round 1 input volume. Template concentration and purity was quantified using NanoDrop™ 2000 and Qubit™ double-stranded DNA (dsDNA) Broad Range (BR) Assay (Thermo Fisher Scientific), and templates were diluted in water to an exact concentration of 2 μg/μL according to Qubit measurements.

Isolation of T cells from Peripheral Blood: Peripheral blood was drawn from healthy donors using an IRB-approved protocol (2018-0103). Blood was collected into Lithium Heparin-coated vacutainer tubes and transferred to 50 ml conical tubes. CD3⁺ primary human T cells were isolated by negative selection as per manufacturer's instructions (Cat #15021, 15061, RosetteSep™ Human T cell Enrichment Cocktail Human T cell Enrichment Cocktail, STEMCELL Technologies, Vancouver, Canada). T cell pellets were resuspended in dilution medium (PBS with 2% FBS) and counted using a hemocytometer with 0.4% Trypan blue viability stain (ThermoFisher Scientific, Waltham, MA). Cells were then resuspended at 1 million/mL in TexMACS™ Cell Culture Medium (Cat #130-097-196, Miltenyi Biotec, Bergisch Gladbach, Germany). T cell cultures were supplemented with 10 ng/ml of IL-7 (Cat #207-IL-005/CF, BioTechne, Minneapolis, MN) and 10 ng/ml of IL-15 (Cat #247-ILB-005/CF, BioTechne, Minneapolis, MN) and stimulated with T cell TransAct™ (10 μL for each mL of culture, Cat #130-111-160, Miltenyi Biotec, Bergisch Gladbach, Germany) for 72 hours respectively.

T cell Electroporation on Lonza 4D-Nucleofector: Following T cell activation, the RNP and DNA were electroporated (EP) into T cells in 16-well EP cuvettes on a 4D-Nucleofector® X Unit (Cat #V4XP-3032, Lonza, Walkersville, VA) using pulse code EH-115. One million cells were electroporated per well in the cuvette. Per reaction, a TRAC locus-specific sgRNA (5′-CAGGGUUCUGGAUAUCUGU-3′; SEQ ID NO: 8) (IDT, Coralville, IA) (2 μL of 100 μM, IDT) was incubated with SpCas9 (0.8 μL of 10 mg/mL, Cat #9212-0.25 MG, Aldevron, Madison, WI) and Poly-L-Glutamic acid (PGA (15,000-50,000 kDa), 1.6 μL of 10 mg/mL solution in DNase free water, Cat #26247-79-0, Millipore Sigma, Burlington, MA) for 15 minutes at 37° C. to form the RNP complex. During incubation, T cells were centrifuged for 300 g×5 min and counted on the Countess® II FL Automated Cell Counter (Thermo Fisher Scientific, Waltham, MA) with 0.4% Trypan Blue viability stain. One million cells per reaction were then aliquoted and spun for 90 g×10 min. Following RNP incubation, linearized dsDNA HDR templates were added to the mixtures (1 μL) in PCR tubes and incubated for at least 5 minutes. Cells were then resuspended in 18.6 μL of P3 buffer (Lonza) and transferred to PCR tubes containing the RNP:DNA mixtures. Contents were then transferred to cuvettes (total volume 24 μL) and electroporated. Immediately following EP, 80 μL of ImmunoCult™ XF (Cat #10981, STEMCELL Technologies, Vancouver, Canada) with no cytokines was added to each reaction which were then rested for 30 minutes at 37° C. Cells were then moved to a 96-flat-bottom well plate containing 160 μL of medium supplemented with 10 ng/ml of IL-7 (BioTechne, Minneapolis, MN), 10 ng/ml of IL-15 (BioTechno, Minneapolis, MN), and 2.5 μM of Nedisertib (M3814) (Cat #S8586, Selleckchem, Houston, TX). Cells were cultured for 24 hours and then transferred to 12-well plates with 1 mL of media and incubated at 37° C. for 48 hours.

T cell Electroporation on Cellares Electroporation System: Following T cell activation, RNPs and DNA were electroporated into T cells in the Cellares Electroporation (CEP) chambers (Cellares, South San Francisco, CA) using electroporation parameters co-developed with Cellares. For each at-scale reaction, 50 million cells were electroporated. Per reaction, a single guide RNA (CAGGGUUCUGGAUAUCUGU; SEQ ID NO: 8) (IDT, Coralville, IA) specific for the TRAC locus (8.33 nmol, IDT) was incubated with SpCas9 (2.05 nmol, Cat #9212-0.25 MG, Aldevron, Madison, WI) for 15 minutes at 37° C. to form the RNP complex. During incubation, T cells were counted on the NucleoCounter® NC-200™ (ChemoMetec Inc, La Jolla, CA), aliquoted (50 million T cells per reaction), and centrifuged at 300 g for 10 min. Following RNP incubation, linearized dsDNA HDR templates were added to the mixtures (222 μg) in PCR tubes and incubated for at least 5 minutes. Cells were then resuspended in 1 mL of Cellares EP Buffer, combined with the RNP:DNA mixtures and transferred to the CEP chambers for electroporation. Cells were rested for 30 minutes at 37° C., then split into two wells of a G-Rex® 6M Well Plate (Cat #80660M Wilson Wolf, Saint Paul, MN) containing 30 mL of medium per well supplemented with 10 ng/ml of IL-7 (BioTechne, Minneapolis, MN), and 10 ng/ml of IL-15 (BioTechne, Minneapolis, MN). Cells were cultured for 3 days and then half the media was removed and replaced with fresh media. The cells were then incubated at 37° C. for 48 hours before being evaluated for editing efficiency.

T cell Electroporation on Thermo Fisher Neon™ NxT Electroporation System: Following T cell activation, the RNP and DNA were electroporated into T cells in Neon™ NxT 10 μL pipette tips (Cat #N1025, Thermo Fisher Scientific) on a Neon™ NXT Electroporation System (Cat #NEONIS, Thermo Fisher Scientific) using pulse code 1600V/10 ms/3. One million cells were electroporated per condition. Per reaction, a TRAC locus-specific sgRNA (5′-CAGGGUUCUGGAUAUCUGU-3′; SEQ ID NO: 8) (0.154 μL of 100 μM, IDT) was incubated with SpCas9 (0.2 μL of 10 mg/mL, Cat #9212-0.25 MG, Aldevron) and PGA (0.123 μL of 10 mg/mL solution in DNase free water, Cat #26247-79-0, Millipore Sigma) for 15 minutes at 37° C. to form the RNP complex. During incubation, T cells were centrifuged for 300 g×5 min and counted on the Countess® II FL Automated Cell Counter (Thermo Fisher Scientific) with 0.4% Trypan Blue viability stain. One million cells per reaction were then aliquoted and centrifuged at 90 g×10 min. Following RNP incubation, linearized dsDNA HDR templates were added to the mixtures (1 μL) in PCR tubes and incubated for at least 5 minutes. Cells were then resuspended in 10 μL of gene editing buffer (Thermo Fisher Scientific) and transferred to PCR tubes containing the RNP:DNA mixtures. Contents were then transferred to Neon™ NxT 10 μL pipette tips and electroporated. Immediately following EP, 80 μL of ImmunoCult™-XF (Cat #10981, STEMCELL Technologies) with no cytokines was added to each reaction which were then rested for 30 minutes at 37° C. Cells were then moved to a 96-flat-bottom well plate containing 160 μL of medium supplemented with 10 ng/ml of IL-7 (BioTechne), 10 ng/ml of IL-15 (BioTechne), and 2.5 UM of Nedisertib (M3814) (Cat #S8586, Selleckchem). Cells were cultured for 24 hours and then transferred to 12-well plates with 1 mL of media and incubated at 37° C. for 48 hours.

T cell Culture: TRAC-CAR T cells were cultured in TexMACS™ media supplemented with IL-7/IL-15 (10 ng/ml) at 1 million cells/mL respectively for the first three days. After EP, TRAC-CAR T cells were cultured in ImmunoCult™ XF for 7 days with IL-7/IL-15 (10 ng/ml). Every 2 days, cells were centrifuged for 300 g×5 min and counted on the Countess® II FL Automated Cell Counter with 0.4% Trypan Blue viability stain. Cells were then resuspended in culture medium at 1 million cells/mL. The same process was repeated on days 5 and 7 post-EP.

Flow Cytometry Analysis: CAR was detected using a 1A7 anti-14G2A antibody (National Cancer Institute, Biological Resources Branch) conjugated to APC using a Lightning Link APC Antibody Labeling kit (Cat #705-0010, Novus Biologicals). TCR was detected using an anti-human TCR a/β antibody conjugate to BV421 (Biolegend, San Diego, CA). Flow cytometry to assess CAR and TCR positivity was performed on Day 8 of manufacturing on an Attune™ NxT flow cytometer (ThermoFisher, Waltham, MA). Immunophenotyping of cells was performed on Day 10 of manufacturing using a spectral immunophenotyping panel on a Cytek® Aurora spectral cytometer (Cytek, California). Briefly, cells were plated in a 96-round bottom well plate (100k for CAR/TCR and 250k for spectral immunophenotyping), washed with 200 u L of PBS and spun at 1200 g×1 min, twice. Cells were then stained for viability with either GhostRed™ 780 (Catalog #50-105-2988, Tonbo Biosciences, San Diego, CA) or Live-Dead™ Blue (Catalog #L23105, ThermoFisher, Waltham MA). For CAR/TCR staining, 1 μL of GhostRed™ 780 was added to 10 mL of PBS to make a stock solution, 100 μL of stock solution was added to each sample and incubated for 30 minutes in the dark. For spectral flow staining, Live-Dead™ Blue stain was resuspended in 50 μL of DMSO, 1 μL added per 1 mL PBS to make a stock solution, and 200 μL of stock solution was added to each sample and incubated for 30 minutes in the dark. After viability stained, samples were washed twice and blocked for 30 minutes with 50 μL FACS buffer (0.5% BSA in PBS) with TruStain FcX™ solution (0.5 L/sample) (Cat #422301, Biolegend, San Diego, CA). Antibodies were then added to 100 μL of BD Brilliant Stain Buffer (Cat #659611, BD Biosciences, Franklin Lakes, NJ) and incubated for 1 hour. Cells were then washed, resuspended in 200 μL or 75 μL of FACS buffer, and analyzed on the Attune™ or Cytek® Aurora respectively. For spectral immunophenotyping, we used CD4, CD8, TCR, and CAR positivity to define populations and for all markers cells were gated by relative size, shape, singlets, viability, TCR negativity and CAR transgene positivity to find an analyzable population of viable CAR T cells.

Stimulation of CAR T Cells and Flow Cytometry: One million Cas9-CLIPT NFAT-mCh or NFAT-15 TRAC-CAR T cells were stimulated in 12-well plates with either PMA/ionomycin (10 ng/mL PMA, 1 μg/mL ionomycin) or 200,000 GD2⁺ neuroblastoma CHLA-20 cells. NFAT-15 were treated with a 1× solution of Brefeldin (Cat #420601, Biolegend, San Dieo, CA) during stimulation. Cells were counted and harvested after 24 hours and stained for flow cytometry. Briefly, 100k cells were plated in a 96-round bottom well plate, washed with 200 μL of PBS and spun at 1200 g×1 min, twice. Cells were then stained for viability with GhostRed™ 780 and CAR in 100 μL of and incubated for 30 minutes in the dark. NFAT-mCh CAR T cells were stained for just CAR and viability, while NFAT-15 CAR T cells treated with the True-Nuclear™ Transcription Factor kit (Car #424401, Biolegend, San Diego, CA) according to the manufacturer's protocol and stained for IL-15 and TGFBR2. Cells were then washed, resuspended in 75 μL of FACS buffer, and analyzed on the Cytek® Aurora instrument and were gated by relative size, shape, singlets, viability, TCR negativity and CAR transgene positivity to find an analyzable population of viable CAR T cells.

In Vitro Cytotoxicity Assay on Incucyte®: 10,000 AkaLUC-GFP CHLA-20 cells were seeded in triplicate on 96-well plates and incubated for 24 hours at 37° C. 24 hours later 100,000, 50,000, 25,000, 12,500, 6,250, or 3130 CAR⁺ T cells from Day 10 of manufacturing were added to each well for effector: target ratios of 10:1, 5:1, 2.5:1, 1.25:1, 0.625:1, or 0.313:1. The plate was centrifuged for 5 minutes at 100 g and then placed in the IncuCyte® S3 Live-Cell Analysis System (Sartorius, Göttingen, Germany) and stored at 37° C., 5% CO₂. Images were taken every 3 hours for 48 hours. Green object count was used to calculate the number of cancer cells in each well and fluorescent images were analyzed with IncuCyte® Base Analysis Software.

Spectral Flow Cytometry Data Analysis: Analysis of spectral flow cytometry data was performed using the Cytek® SpectroFlo program. Single positive controls for each color were collected and analyzed in SpectroFlo for positive and negative populations. SpectroFlo's unmixing algorithm was then used to compensate for spillover and autofluorescence of cells. Data was then exported to FlowJo (V10.9.0) where samples were gated for non-debris, singlets, and live cells. TCR, and CAR positivity were used to gate cell populations for in vitro samples. Representative plots were generated in FlowJo using fluorescence minus one controls to set positive gates.

Linearization Gel Assays: TRAC single guide RNA (1 μL of 100 μM) was incubated with SpCas9 (0.8 μL of 10 mg/mL) with or without poly-L-glutamic acid (1.6 μL of 10 mg/mL solution in DNase free water) for 15 minutes at 37° C. to form the RNP complex. To RNP complexes with and without PGA, Cas9-CLIPT or Control Nanoplasmid™ was added and incubated for 10 minutes. RNP: Nanoplasmid™ complexes were then run on a 1% agarose gel with a 1 kb ladder.

Data Analysis and Software: All data analyses were performed in GraphPad Prism (V.10.0.2) and Microsoft Excel. Statistical tests were done in GraphPad Prism and indicated in the figure legends. Nanoplasmid™ sequences were designed in Benchling. FlowJo was used to analyze.fcs files exported from Cytek® SpectroFlo and Cytek® Attune NxT software. Representative flow plots were exported from FlowJo. Figures were created and organized using Adobe Illustrator (V28.0). A p value less than 0.05 was defined as significant.

DNA Extraction and PCR Amplification: Genomic DNA was extracted from each sample using DNA QuickExtract™ (Lucigen) and the following program: 65° C. for 15 min, 68° C. for 15 min, and 98° C. for 10 min. Amplicon primer sequences were designed in Benchling to flank the homology arms of the donor DNA templates. PCR was performed according to the manufacturer's instructions using Q5® Hot Start Polymerase (NEB) using the following programs for expected amplicon sizes-3 kb amplicon: 98° C. (30 s), 30 cycles of 98° C. (10 s), 68° C. (30 s), 72° C. (1:30 min), and a final extension at 72° C. (2 min); 6 kb amplicon: 98° C. (30 s), 40 cycles of 98° C. (10 s), 68° C. (30 s), 72° C. (5 min), and a final extension at 72° C. (5 min).

Forward Primer:
SEQ ID NO: 9
5′-GGCCTTTTTCCCATGCCTGCCT-3′,
Reverse Primer:
SEQ ID NO: 10
5′-TGCCCTCTCCTGCCACCTTCTC-3′,

Oxford Nanopore Sequencing: DNA concentration of PCR samples was quantified using NanoDrop™ 2000 and Qubit™ double-stranded DNA (dsDNA) Broad Range (BR) Assay (Thermo Fisher Scientific), and PCR samples were diluted in nuclease-free water to an exact concentration of 30 ng/μL according to Qubit™ measurements. Samples were sequenced on the Oxford Nanopore Technologies PromethION™ 24 according to the manufacturer's instructions. Prior to read alignment, the reference sequence was filtered from all non-IUPAC characters. All variants exhibiting a FILTER status of PASS are merged into the consensus sequence. Passing reads were sorted using a 2:1 weighted preference (length: quality) and the top 90% are kept for further analysis. QUAL is the Phred-scaled probability that the site has no variant and is computed as QUAL=−10*log 10 (posterior genotype probability of a homozygous-reference genotype (GT=0/0)). Analysis was performed using Integrative Genomics Viewer (IGV) and CRISPResso2, crispresso2.pinellolab.org.

Whole Genome Sequencing for Off-Target Sites: Genomic DNA was extracted from Cas9-CLIPT CAR T cells using the Gentra® Puregene kit (Qiagen, Germantown, MD) per the manufacturer's instructions, and quantified using Qubit. Samples were sequenced on the Oxford Nanopore Technologies PromethION 24 according to the manufacturer's instructions. Whole genome sequencing reads were aligned to the scFv region of the CAR transgene; reads with alignment to the transgene were subsequently mapped to the human genome (GRCh38). Mapping was performed with minimap2 utilizing first half of the detectIS pipeline. Reads were filtered for a minimum alignment length to chromosome or CAR of 150 bp, and a minimum MAPQ value of 30. Integration sites were identified from reads aligning to both the transgene and the human genome filtered with an overlap window threshold (distance between the transgene aligning and the human genome aligning segments/total length of read) of 0.5.

### Example 1: Manufacturing Cas9-CLIPT TRAC-CAR T Cells

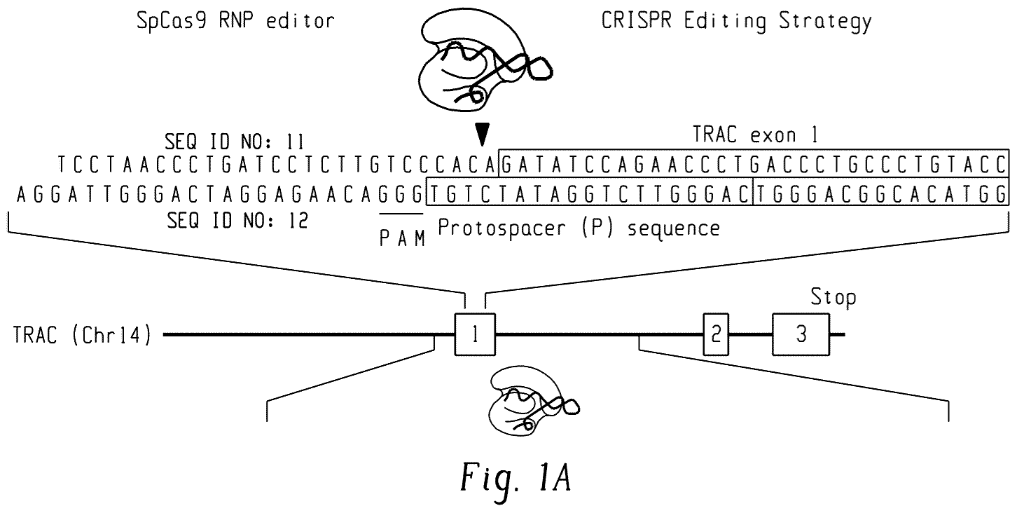

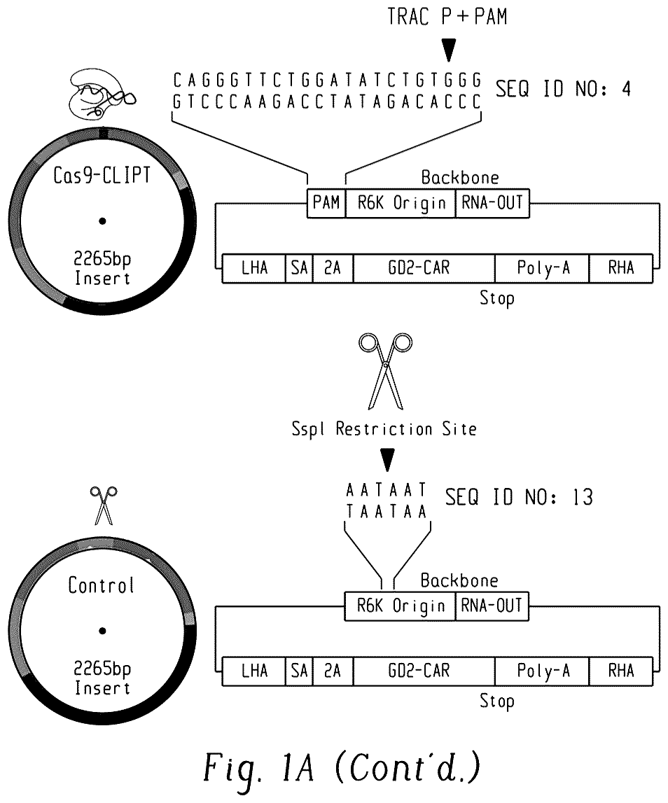

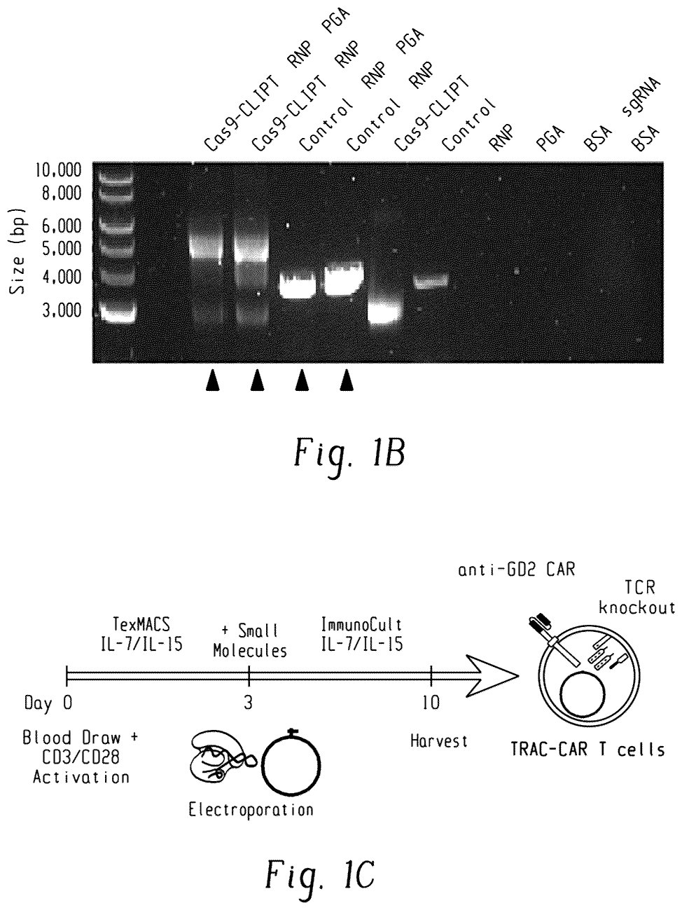

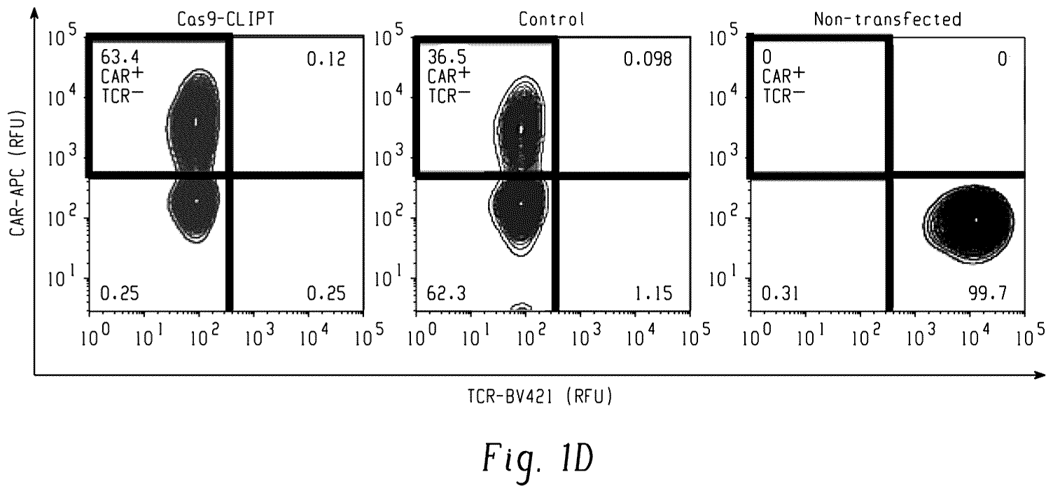

TRAC-CAR T cell manufacturing should yield robust numbers of CAR⁺ cells and include well-characterized manufacturing of ancillary materials to meet GMP requirements. A single target sequence consisting of a protospacer sequence (P) together with the protoadjacent motif sequence (PAM) for a TRAC single guide RNA (sgRNA) into a Nanoplasmid™ donor template was explored to streamline non-viral CAR T cell manufacturing. Two different HDR templates containing an anti-GD2 CAR were constructed: 1) a circular Cas9-CLIPT template that can be linearized by an RNP with TRAC sgRNA; and 2) a “Control” circular plasmid that can be linearized via an Sspl restriction site (FIG. 1A). The P+PAM sequence was inserted upstream of the left homology arm in an orientation with the PAM, such that Cas9 cleavage would produce a linearized plasmid, containing the plasmid backbone downstream of the right homology arm. In this design, any unintended integration of the backbone through HDR-independent DNA repair would not disrupt the transcription of the CAR.

The two HDR templates were prepared and a gel shift assay was performed, with and without the RNP, and poly-L-glutamic acid (PGA) which can be added to the electroporation buffer for T cells. After 10 minutes of incubation with RNP, the Cas9-CLIPT Nanoplasmid™ was linearized, regardless of PGA addition (FIG. 1B). The Cas9-CLIPT samples migrated slower within the electrophoresis gel, indicating a larger molecular weight for the Cas9-CLIPT molecular complex than the Sspl-linearized Control Nanoplasmid™. The slow migration depended on the sgRNA sequence, as the Cas9-CLIPT co-incubated with a RNP with a nontarget sgRNA failed to migrate slowly (data not shown). These results indicate that Cas9-CLIPT forms a complex with the RNP, based upon the sgRNA sequence, and becomes linearized within minutes of incubation with a matching RNP.

The first time the gel shift assay was performed, the Control Nanoplasmid underwent one freeze/thaw and showed one band of the correct size (data not shown). After the Control Nanoplasmid and Cas9-CLIPT Nanoplasmid underwent multiple freeze/thaws, another gel shift assay was performed, and two bands were observed for Control Nanoplasmid: one at the correct size of the linear Control Nanoplasmid and one smaller band (data not shown). In the second gel shift assay, only one correct size band was still observed for Cas9-CLIPT Nanoplasmid. After multiple freeze/thaws the Control Nanoplasmid becomes a heterogeneous template while the Cas9-CLIPT Nanoplasmid remains a homogeneous template.

To examine whether Cas9-CLIPT could be used to engineer a therapeutically relevant CAR T cell, T cells were electroporated with Nanoplasmid™ templates and a sgRNA targeted to the TRAC locus complexed within a SpCas9 RNP. The cells were metabolically primed with a 3-day activation in TexMACS™ media and expanded for 7 days in ImmunoCult™-XF with IL-7/IL-15 post-electroporation (EP) (FIG. 1C). The use of either TexMACS™ or ImmunoCult™-XF media alone did not significantly impact the knock-in rate (data not shown). 2 μg of Cas9-CLIPT donor template and Control per 1 million cells was used as this optimized concentration preserved high knock-in while maintaining viability and cell expansion (data not shown). Thirty minutes post-EP, cells were incubated with the DNA-PK inhibitor, Nedisertib (M3814), post-EP to block NHEJ and promote HDR DNA repair (data not shown). On day 5 post-EP, there was a 1.7-fold increase in CAR positivity when using Cas9-CLIPT as the donor template (Cas9-CLIPT: 61% (3), Control: 36% (1), p<0.001)) versus Control TRAC-CAR T cells. PGA and M3814 were used to further increase knock-in efficiencies. Greater than 98% of Cas9-CLIPT and Control TRAC-CAR T cells lacked expression of the endogenous TCR (Cas9-CLIPT: 99.5 (0.2), Control: 99.2 (0.5)) (FIG. 1D). Non-transfected cells are T cells that did not receive any treatment during manufacturing; they were cultured according to our manufacturing process. We produced Control TRAC-CAR T cells using a circular Control Nanoplasmid instead of linearizing the Control Nanoplasmid prior to EP, though the linear Control Nanoplasmid resulted in higher CAR knock-in efficiencies (data not shown). A PCR-generated tCTS dsDNA donor template was also tested, and it was found that the Cas9-CLIPT Nanoplasmid had improved CAR knock-in efficiencies compared to the PCR-generated tCTS dsDNA donor template at all DNA doses.

The addition of poly-L-glutamic acid (PGA) during RNP incubation and the addition of Nedisertib during post-EP T cell recovery further increased knock-in efficiencies. Greater than 98% of Cas9-CLIPT and Control TRAC-CAR T cells lacked expression of the endogenous TCR (Cas9-CLIPT: 99.5% (0.2%), Control: 99.2% (0.5%)) (FIG. 1E). The percentages of CAR knock-in (Cas9-CLIPT: 62% (2%), Control: 37% (1%)) and TCR knockout (Cas9-CLIPT: 99.4% (0.3%), Control: 99.0% (0.6%); p<0.001) also remained unchanged until day 7 post-EP (FIG. 1E). There were no significant differences between Cas9-CLIPT and Control TRAC-CAR T cell fold-expansion (Cas9-CLIPT: 8.8 (3.5), Control: 10.0 (3.2), RNP Only: 5.6 (1.0), No EP: 22 (6.5)) or viability (Cas9-CLIPT: 89.7% (3.8%), Control: 88.8% (5.6%), RNP Only: 85.8% (3.3%), No EP: 93% (2.3%)) after manufacturing (FIG. 1F). Though the EP of T cells and addition of donor template to both Cas9-CLIPT and Control-TRAC CAR T cells hindered T cell fold-expansion and viability compared to T cells electroporated with no donor template (RNP Only) and T cells that were not electroporated but were incubated with Cas9-CLIPT Nanoplasmid (No EP) (FIG. 1F).

Since the transport of Cas9-CLIPT into T cells could depend on the mechanics of the electroporation, we adapted our workflow on two other electroporation devices relevant to clinical scale manufacturing: the Cellares Electroporation (CEP) system and the Thermo Fisher Scientific Neon™/Xenon™ system. The CEP system affords users fine control over key electroporation parameters and operates in batch mode to enable electroporation at a clinical scale. In this system, a small-scale EP that uses 5 million T cells resulted in Cas9-CLIPT CAR T cells with knock-in rates well above Control conditions (CAR Knock-in: Cas9-CLIPT: 26% (8), Control: 5%; TCR Knockout: Cas9-CLIPT: 88% (2), Control: 90%) (FIG. 1G). For a 50 million T cell EP using the CEP system (large-scale), CAR knock-in efficiency was 15% compared to 26% in small-scale CEP experiments (CAR Knock-in: 15% (8%); TCR Knockout: 86% (2%)) (FIG. 1G). Further optimization on the CEP system may enhance clinical-scale approaches. Cas9-CLIPT strategies also improved knock-in using the ThermoFisher Scientific system at a small scale (data not shown). These results demonstrate that the Cas9-CLIPT Nanoplasmid™ can increase knock-in in various EP conditions on multiple EP instruments.

### Example 2: On-Target Genomic Analysis of Cas9-CLIPT TRAC CAR T Cells

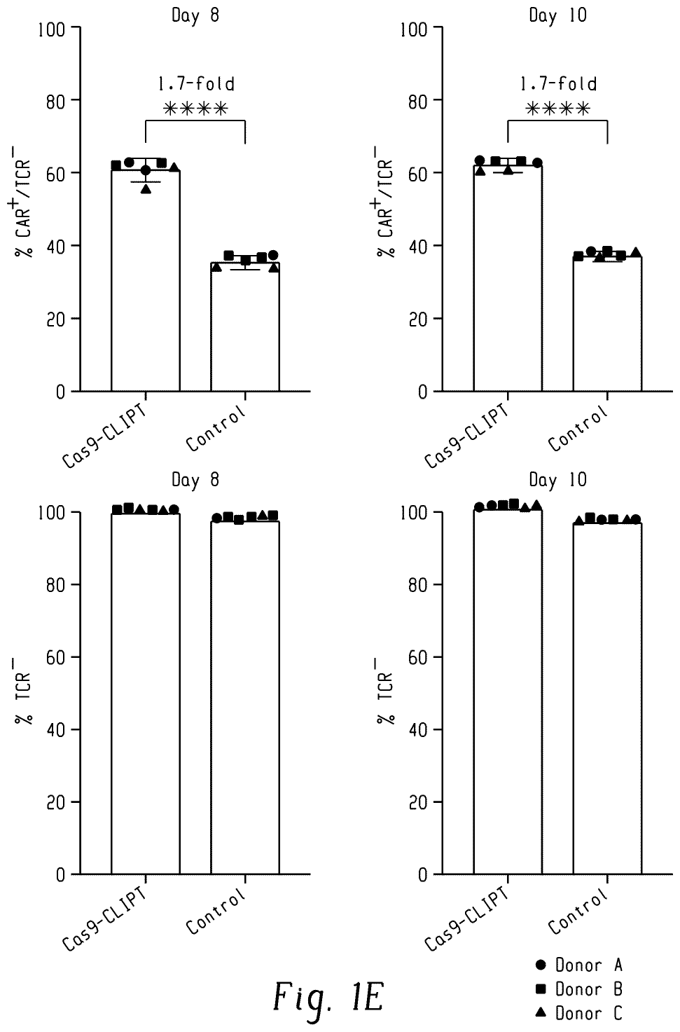

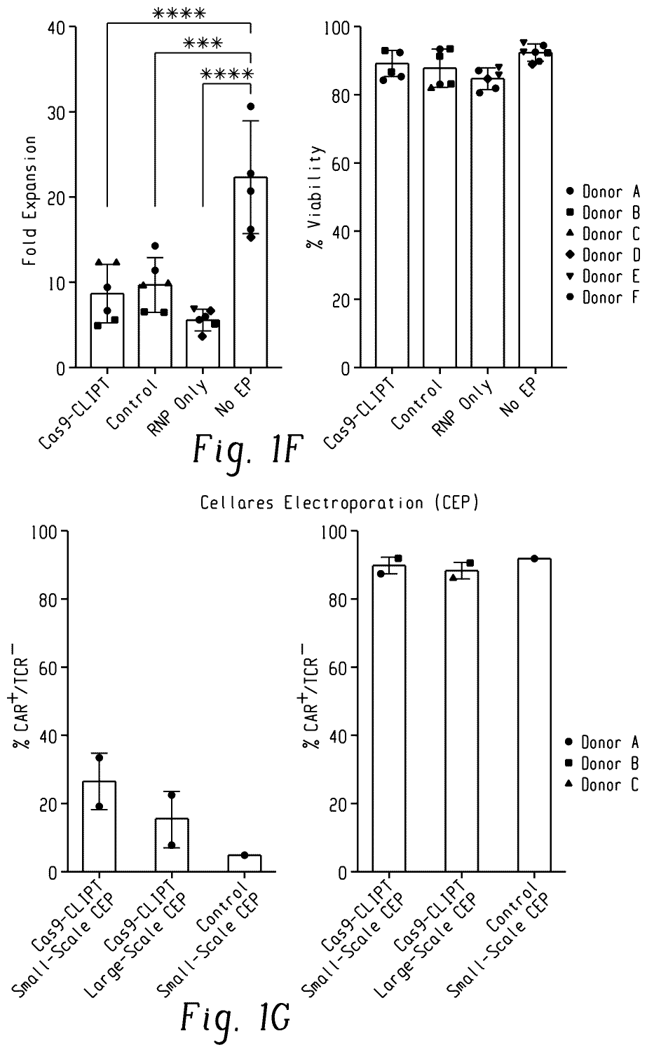

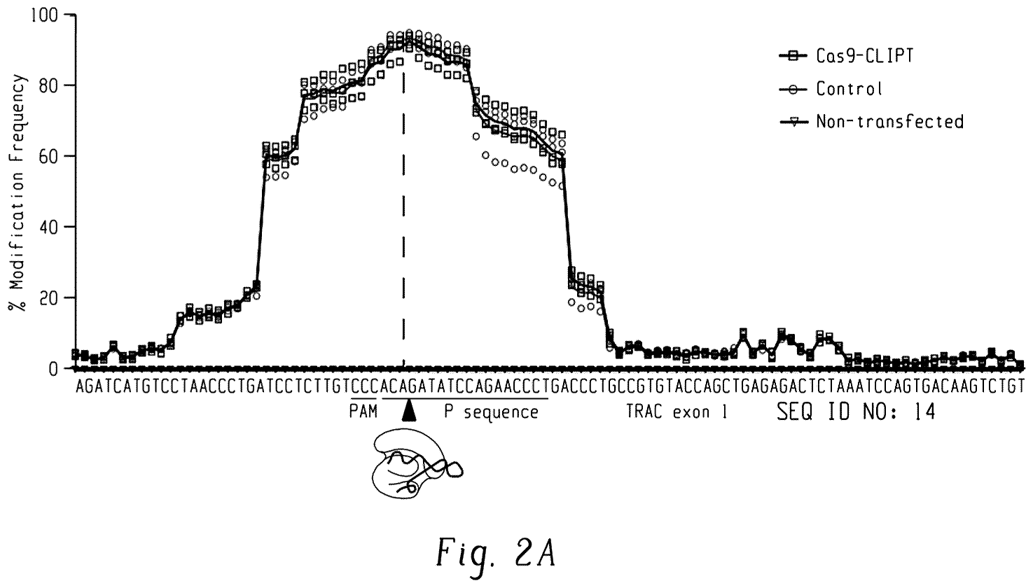

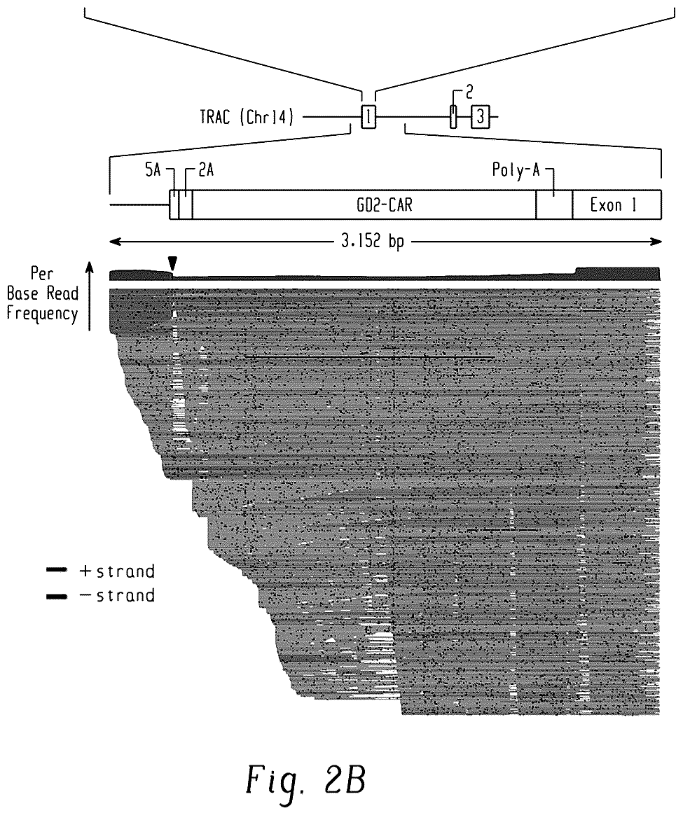

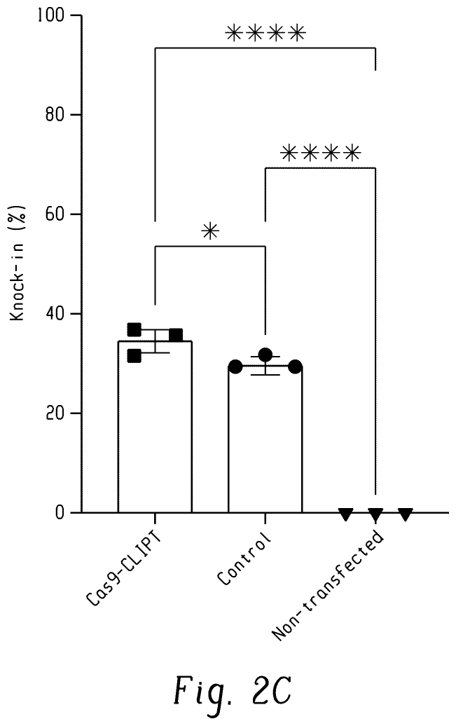

The insertion of Cas9-CLIPT donor templates into the TRAC locus was confirmed by performing long-read sequencing on genomic DNA isolated from the cell product. DNA was isolated during day 10 of manufacturing from Cas9-CLIPT and Control TRAC-CAR T cells from three different donors, the 3 kb regions of interest amplified via PCR around the intended insertion site, and amplicons were sequenced using an Oxford Nanopore instrument. The modification frequency of each base pair around the TRAC cut site relative to the wild-type sequence was calculated for Cas9-CLIPT and Control TRAC-CAR T cell amplicons by dividing by the number of reads not aligned to the wild-type sequences at each base pair with the total number of reads observed at each base pair. High on-target genome editing was observed, as indicated by high indel formation precisely at the TRAC target site (FIG. 2A). There were no significant differences in the indel spectrum between Cas9-CLIPT and Control edited cells (FIG. 2A). These alleles are predicted to result in a TCR knockout through frameshifts in the coding sequence and nonsense-mediated decay of the resulting TRAC mRNA transcripts. Notably, the alleles with full-length CAR insertion were robustly observed in the reads from sequencing (FIG. 2B). An exact match to the GD2-CAR sequence throughout the transgene and at the junctions of the homology arms was seen (FIG. 2B). These results are consistent with HDR-mediated precise knock-in of the desired CAR transgene sequences. The allelic knock-in efficiency was estimated by dividing the number of reads aligned to the insert by the total number of reads aligned to both the insert and the unedited sample and found a higher percentage of precise insertions with Cas9-CLIPT (Cas9-CLIPT: 35%, Control: 30%, p=0.046) (FIG. 2C).

While the allelic knock-in efficiency was estimated by dividing the number of reads aligned to the insert by the total number of reads aligned to both the edited and the unedited samples, and a higher percentage of precise insertions with Cas9-CLIPT (FIG. 2C), the allelic knock-in efficiencies are lower than the observed flow cytometry results (FIG. 1E). This may be due to PCR bias. Since the CAR T cell products are not 100% efficient, there are still edited and unedited T cells within one sample. When attempting to perform PCR on one sample, the DNA from unedited cells will produce a shorter PCR product than the DNA from edited cells due to the size of the transgene insert. Due to PCR bias favoring smaller amplicons during annealing and extension steps, it is easier for the smaller, unedited DNA fragments to get amplified during PCR than the larger, edited DNA fragments. Thus, it is possible the allelic knock-in efficiencies are not as high as the flow cytometry knock-in efficiencies because it was easier to amplify the unedited DNA than the edited DNA.

### Example 3: Off-Target Genomic Analysis of Cas9-CLIPT TRAC-CAR T Cells

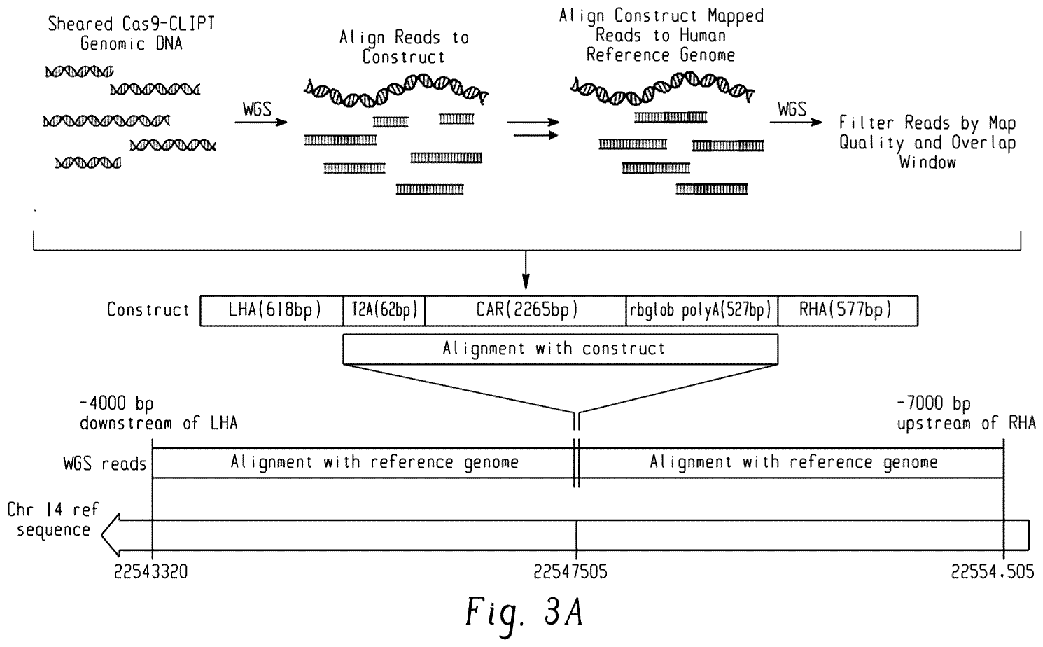

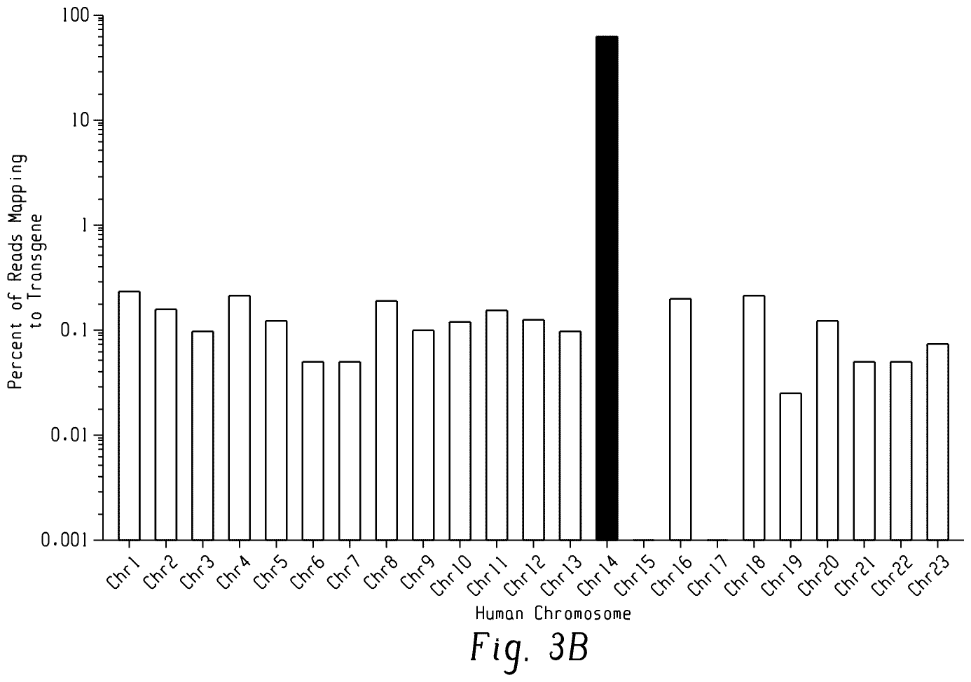

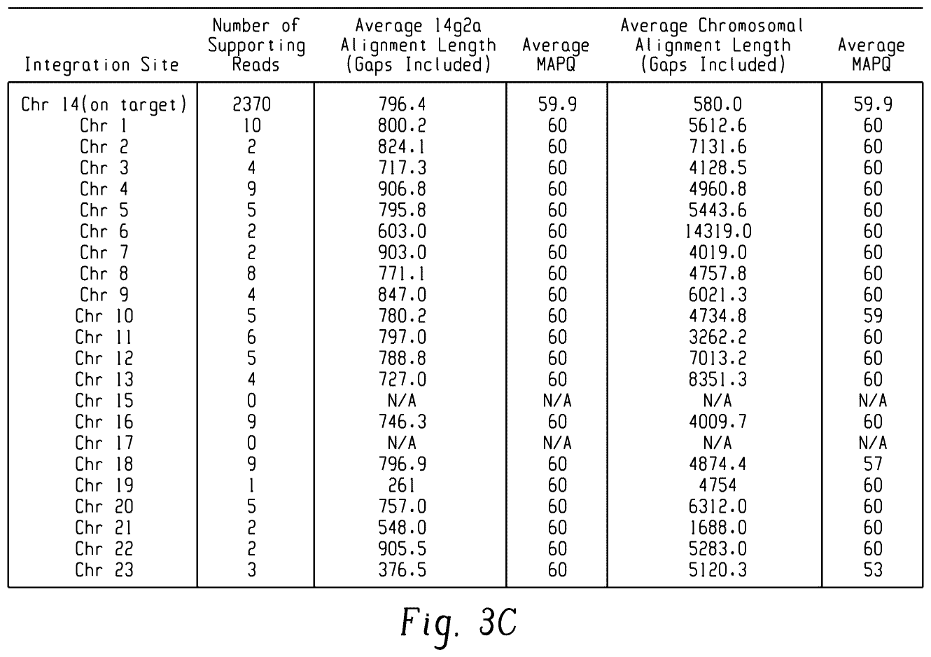

Though CRISPR/Cas9 is a precise genome editing tool, it is possible that the sgRNA used to edit the TRA C locus of T cells could bind elsewhere in the human genome, causing unintended off-target edits. To determine if any off-target sites exist in Cas9-CLIPT TRAC-CAR T cells, whole genome sequencing (WGS) was performed (FIG. 3A). WGS allows for unbiased tracking of integration sites of our transgene since WGS does not have PCR amplification biases nor does it require the nomination of off-target sites. Genomic DNA was prepared for long-read nanopore sequencing via Oxford Nanopore Technologies and aligned to a reference sequence containing the CAR transgene. Successful mapping to the reference human genome provided 30× coverage of the genome. On-target integration was observed in 3270 of 3367 reads (97.3%) with alignment to the CAR transgene (FIG. 3B). Off-target integration was minimally observed in all but two human chromosomes (FIG. 3B). Some of the off-target hits are likely hits because the scFv of the CAR transgene aligns with the immunoglobulin loci found in chromosomes 2 and 22. Off-target integration sites were identified from reads that aligned to both the transgene and the human genome if there was a minimum alignment length of 150 bp and a minimum MAPQ value of 30 (FIG. 3C). Overall, the few number of reads aligned at off-target sites is likely not cause for concern for off-target integration when using Cas9-CLIPT Nanoplasmid.

### Example 4: Phenotypic Analysis of Cas9-CLIPT TRAC-CAR T Cells

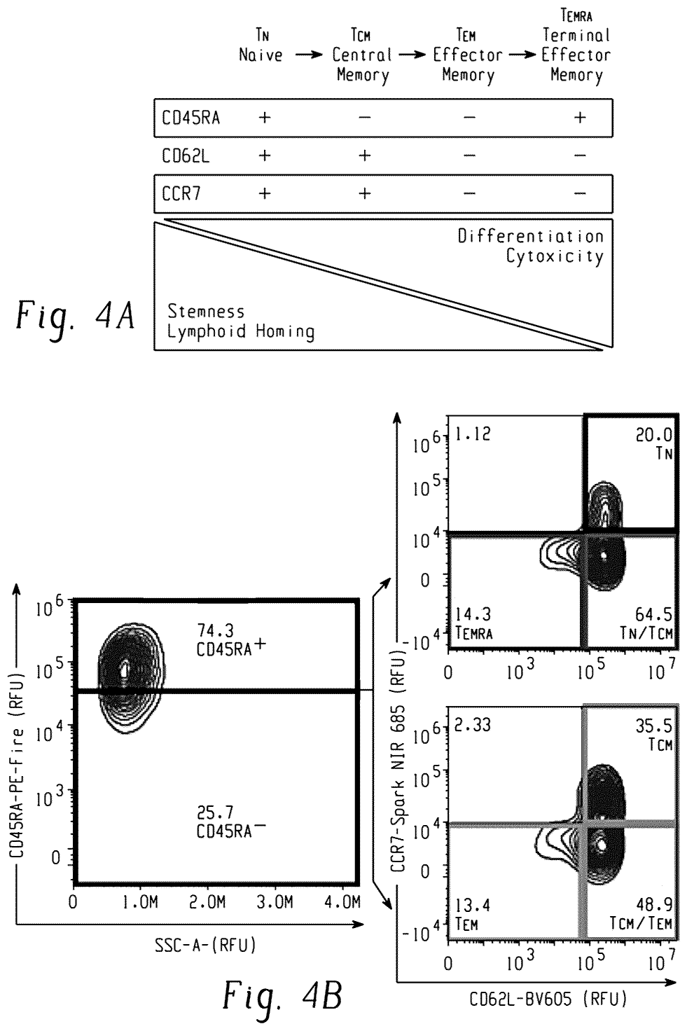

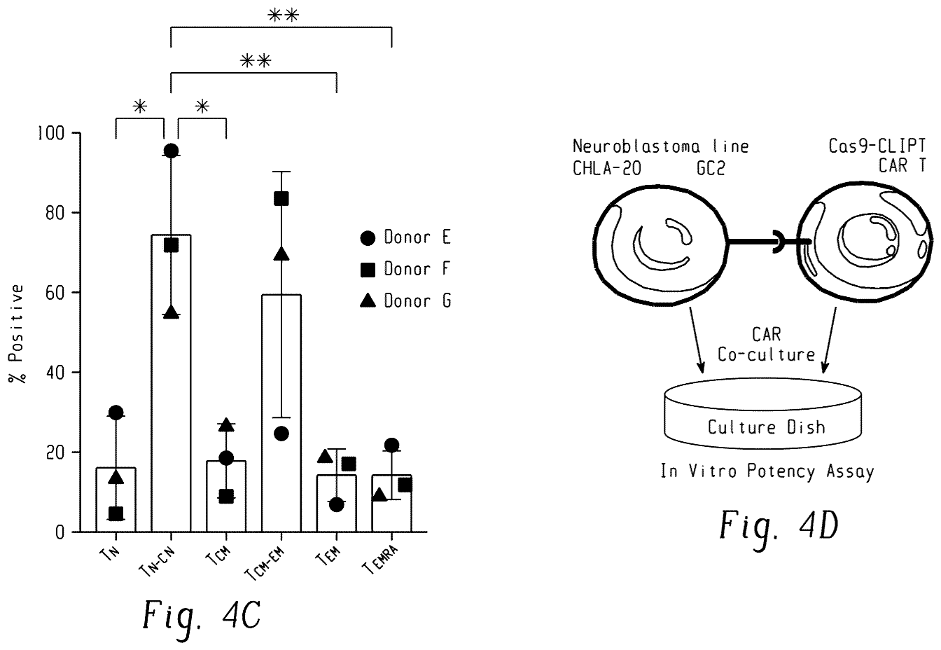

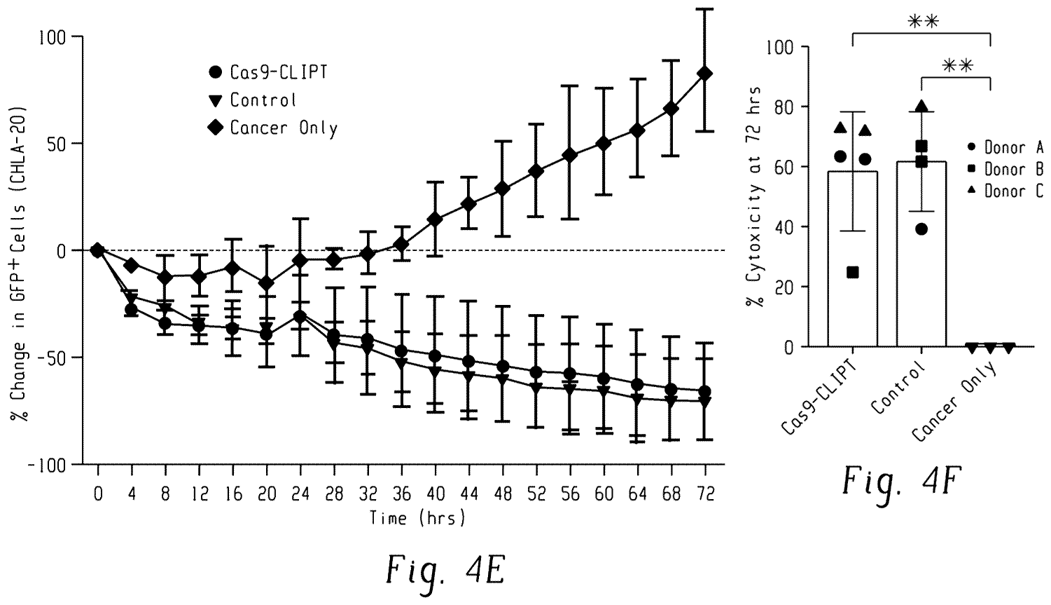

Ex vivo culture of CAR T cells with artificial activation and high concentrations of cytokines can trigger differentiation into short-lived effector cells that struggle to persist in vivo. In contrast, preserving the naïve, stem cell memory T cell population during manufacturing can increase persistence. Control TRAC-CAR T cells undergoing ‘metabolic priming’ have improved stem cell memory properties. Cas9-CLIPT TRAC-CAR T cells cultured this way were analyzed for a similar phenotype. Spectral flow cytometry was utilized on the cell product to distinguish the CAR⁺/TCR population of Cas9-CLIPT TRAC-CAR T cells and profile the surface markers for CD8, CD4, and stem cell memory (data not shown), such as CD45RA, CCR7, and CD62L. These stem cell memory markers decrease as T cells differentiate into effector cells (FIG. 4A). 59% (11) of the Cas9-CLIPT TRAC-CAR were positive for cytotoxic CD8 (data not shown), indicating a balanced CD8 and CD4 set of T cells within the product. In addition, T cells can also be classified as Naïve (T_{N}) (CD45RA⁺/CD62L⁺/CCR7⁺), Naïve-Central Memory (T_{N-CM}) (CD45RA⁺/CD62L⁺/CCR7⁻), Central Memory (T_{CM}) (CD45RA⁻/CD62L⁺/CCR7⁺), Central-Effector Memory (T_{CM-EM}) (CD45RA⁻/CD62L⁺/CCR7⁻), Effector Memory (T_{EM}) (CD45RA⁻/CD62L⁺/CCR7⁻), or terminal effector (T_{EMRA}) (CD45RA+/CD62L-/CCR7⁻). Cas9-CLIPT TRAC-CAR T cells were 8% (6) T_{N}, 37% (10) T_{N-CM}, 9% (5) T_{CM}. 30% (15) T_{CM-EM}, 7% (3) T_{EM}, and 7% (3) T_{EMRA} respectively across three biological donors, indicating a large portion of naïve T cells in the pre-infusion product (T_{N-CM} vs T_{N}, T_{CM}, T_{EM}, or T_{EMRA}; p=0.010, 0.013, 0.008, or 0.0083) (FIG. 4B, C).

### Example 5: Potency of Cas9-CLIPT TRAC-CAR T Cells

The potency of Cas9-CLIPT TRAC-CAR T cells was evaluated against the GD2⁺ neuroblastoma cell line, CHLA-20, by measuring cytotoxicity after co-culture. 10,000 CHLA-20 target cells were seeded in 96-well plates and cultured for 24 hours, after which Cas9-CLIPT and Control TRAC-CAR T cells were added at 10:1, 5:1, 2.5:1, and 1.25:1 effector: target (E:T) ratios (FIG. 4D). All groups successfully lysed CHLA-20 cells over 72 hours at each E:T ratio (FIG. 4E) with no differences in the extent of cytotoxicity for a 1.25:1 E:T after 72 hours of co-culture (Cas9-CLIPT: 66% (22), Control: 70% (19), Cancer Only 0% (0), p (Cas9-CLIPT or Control vs Cancer Only)=0.006 or 0.005) (FIG. 4F). These results demonstrate that our Cas9-CLIPT integration strategy can create potent TRAC-CAR T cells.

### Example 6: Large Transgene Knock-In Using Cas9-CLIPT

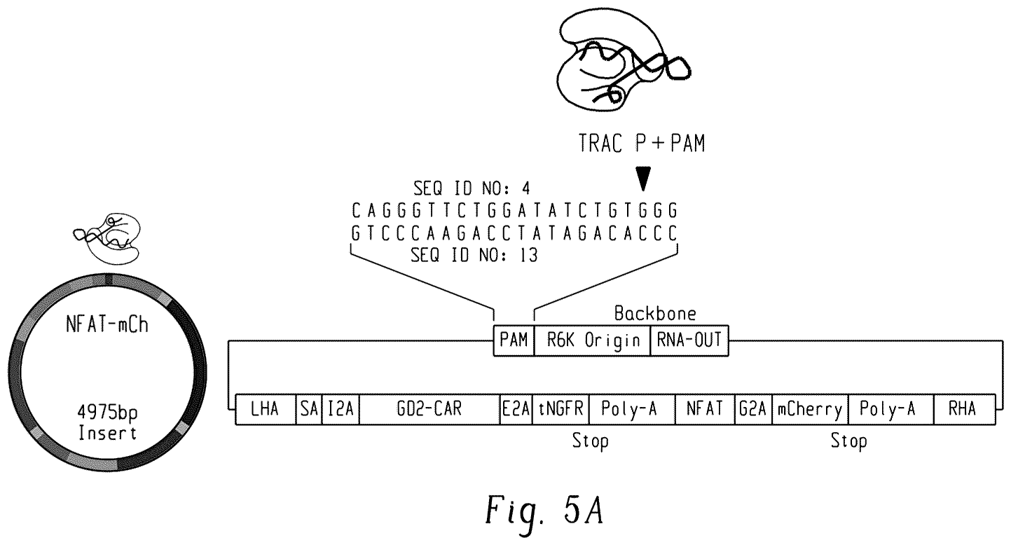

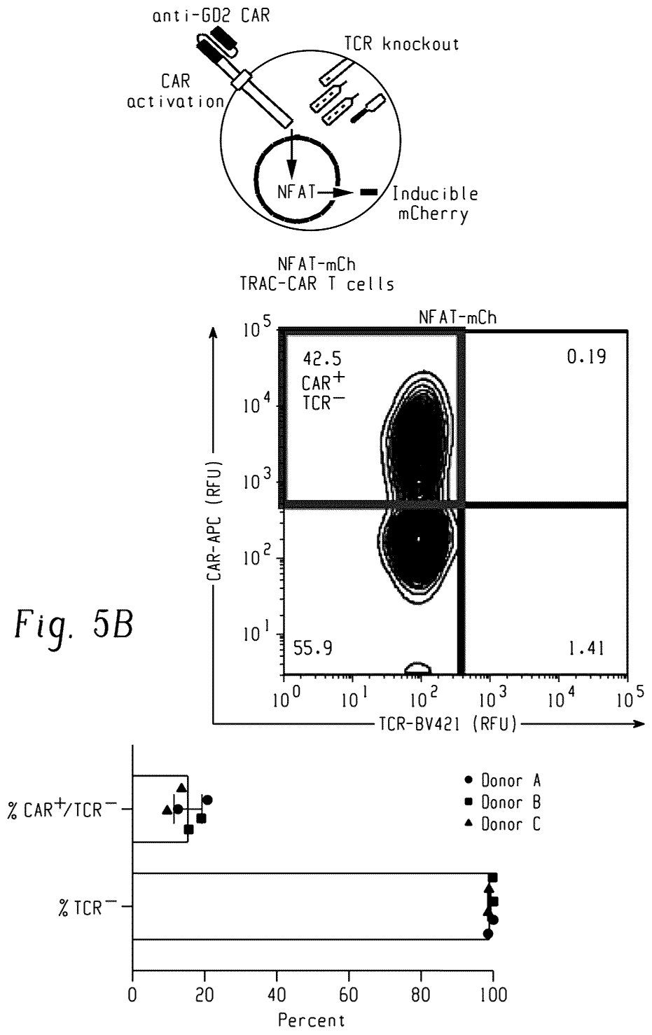

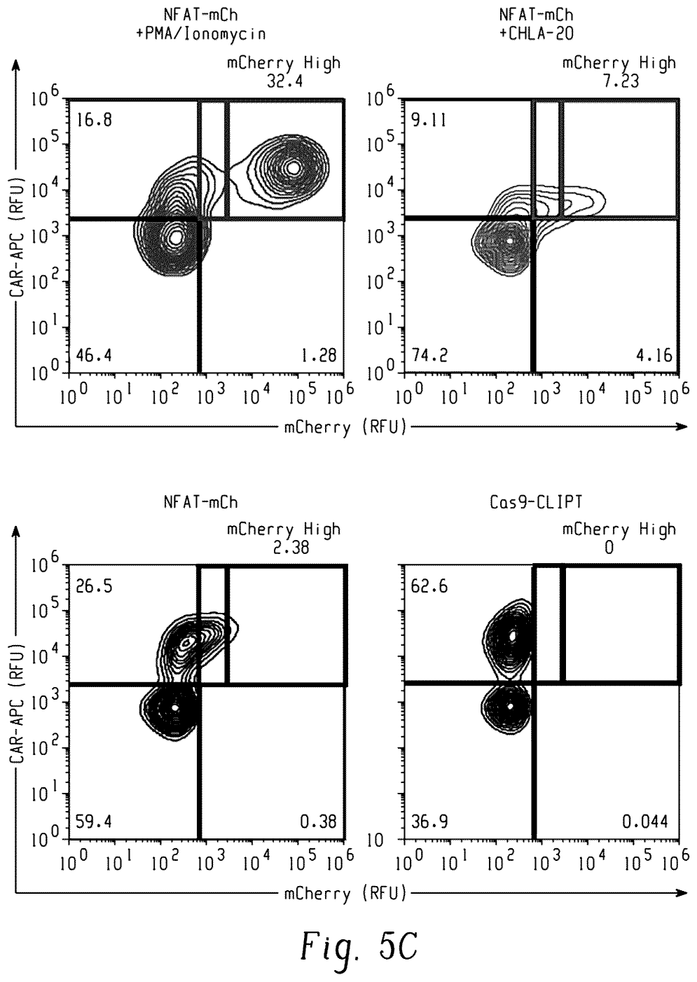

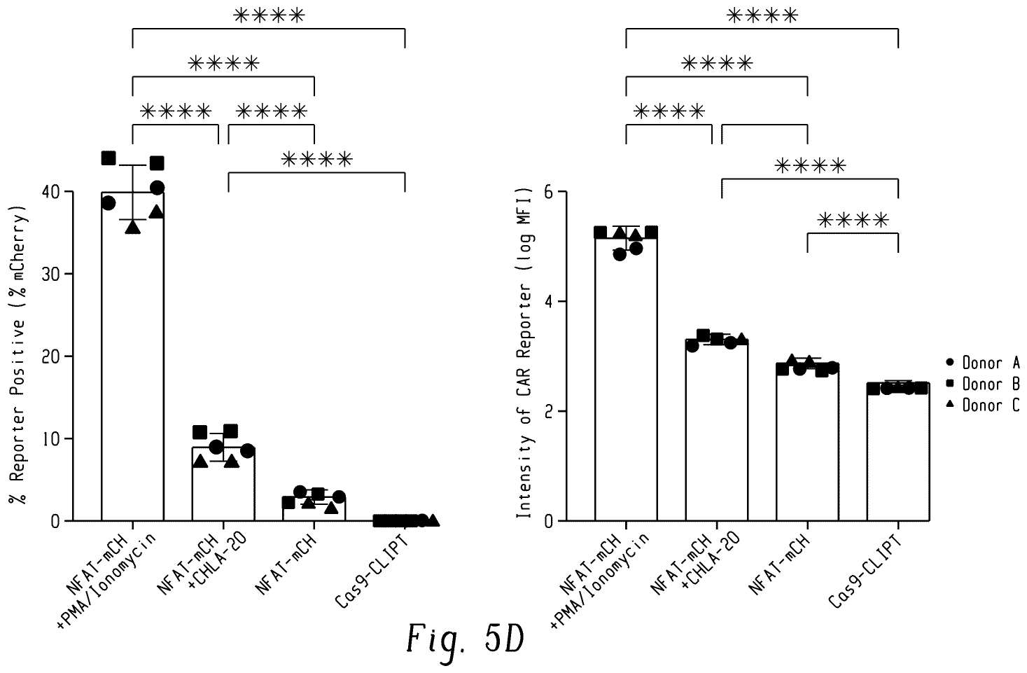

The immune response of T cells to intracellular circular plasmid dsDNA can limit the knock-in of large (>2 kb) transgenes. To explore whether Cas9-CLIPT strategies could be adapted to larger transgenes, a circular Cas9-CLIPT NFAT-mCh plasmid was constructed. In addition to containing an anti-GD2 CAR within the donor template, the transgene insert sequence contains an NFAT (nuclear factor of activated T cells) response element with minimal IL-2 promoter to drive expression of a downstream fluorescent mCherry reporter protein (FIG. 5A). NFAT is a key transcription factor that initiates transcription of IL-2 during T cell activation through signaling from the endogenous TCR or an integrated CAR. Using the large Cas9-CLIPT NFAT-mCh, high knock-in efficiency was observed via flow cytometry assays (Cas9-CLIPT NFAT-mCh Knock-in: 38% (4)) and minimal TCR expression (Cas9-CLIPT NFAT-mCh TCR Knockout: 99% (0.2)) was also observed (FIG. 5B), indicating that the Cas9-CLIPT strategy can successfully edit T cells with >5 kb gene insertions. RNP incubation with PGA prior to addition of NFAT-mCh Nanoplasmid significantly increased CAR knock-in and TCR knockout efficiencies and may be useful for clinically-relevant efficiencies of NFAT-mCh TRAC-CAR T cells (data not shown)

To demonstrate that the integrated construct was functional, Cas9-CLIPT NFAT-mCh and Cas9-CLIPT TRAC-CAR T cells were stimulated with either PMA-ionomycin or the GD2⁺ neuroblastoma cell line, CHLA-20. First, flow cytometry was performed after 24 hours to measure the NFAT-driven expression of mCherry in CAR⁺ T cells. Stimulation of T cells with either method successfully induced expression of mCherry to different extents in CAR⁺ Cas9-CLIPT NFAT-mCh T cells, which was gated into ‘high’ and ‘low’ fractions (mCherry High and mCherry Low) in the CAR⁺/mCherry⁺ population. (FIG. 5C). The low gate includes some mCherry expression driven by NFAT in the absence of stimulation, while the high gate is specific to antigen or PMA/ionomycin stimulation.

Stimulated NFAT-mCh CAR T cells had significantly higher percentages of mCherry High cells than unstimulated Cas9-CLIPT NFAT-mCh or Cas9-CLIPT CAR T cells (Cas9-CLIPT NFAT-mCh+PMA/ionomycin: 32.1% (2.7), Cas9-CLIPT NFAT-mCh+CHLA-20:7.2% (1.4), Cas9-CLIPT NFAT-mCh: 1.6% (0.4), Cas9-CLIPT: 0.01% (0.02), p (Cas9-CLIPT NFAT-mCh+PMA-ionomycin or CHLA-20 vs Cas9-CLIPT NFAT-mCh or Cas9-CLIPT)<0.001, p (Cas9-CLIPT NFAT-mCh+PMA/ionomycin vs CHLA-20)<0.0001) (FIG. 5D). Unstimulated Cas9-CLIPT NFAT-mCh CAR T cells also had higher expression of mCherry Low cells than those stimulated with PMA-ionomycin or CHLA-20 cells indicating a low level activation of NFAT (Cas9-CLIPT NFAT-mCh+PMA/ionomycin: 2.0% (0.5), Cas9-CLIPT NFAT-mCh+CHLA-20: 5.6% (0.4), Cas9-CLIPT NFAT-mCh: 11.7% (2.7), Cas9-CLIPT: 0.5% (0.1), p (Cas9-CLIPT NFAT-mCh vs Cas9-CLIPT NFAT mCh+PMA-ionomycin or CHLA-20 cells or Cas9-CLIPT)<0.001, p (Cas9-CLIPT NFAT-mCh+CHLA-20 vs Cas9-CLIPT NFAT mCh+PMA-ionomycin or Cas9-CLIPT)<0.001 and =0.002 (data not shown). Antigen-stimulated NFAT-mCh CAR T cells had higher mCherry median fluorescence intensity (MFI) than unstimulated cells, showing higher mCherry expression within the CAR⁺ population (Cas9-CLIPT NFAT-mCh+PMA-ionomycin: 4.7 (0.2), Cas9-CLIPT NFAT-mCh+CHLA-20:3.1 (0.1), Cas9-CLIPT NFAT-mCh: 2.7 (0.1), Cas9-CLIPT: 2.4 (0.01), p (Cas9-CLIPT NFAT-mCh+PMA-ionomycin vs Cas9-CLIPT NFAT-mCh+CHLA-20 or Cas9-CLIPT NFAT mCh+ or Cas9-CLIPT)<0.001, p (Cas9-CLIPT NFAT-mCh+CHLA-20 vs Cas9-CLIPT NFAT mCh+ or Cas9-CLIPT)<0.001, p (Cas9-CLIPT NFAT-mCh vs Cas9-CLIPT)<0.001) (FIG. 5D). Stimulation by GD2 antigens on the CHLA-20 line resulted in weaker mCherry expression versus PMA-ionomycin stimulation, as the former had a higher percentage of mCherry Low cells and correspondingly a lower percentage of mCherry-High cells (FIG. 5C).

### Example 7: On-Target Genomic Analysis of NFAT-mCh TRAC-CAR T Cells

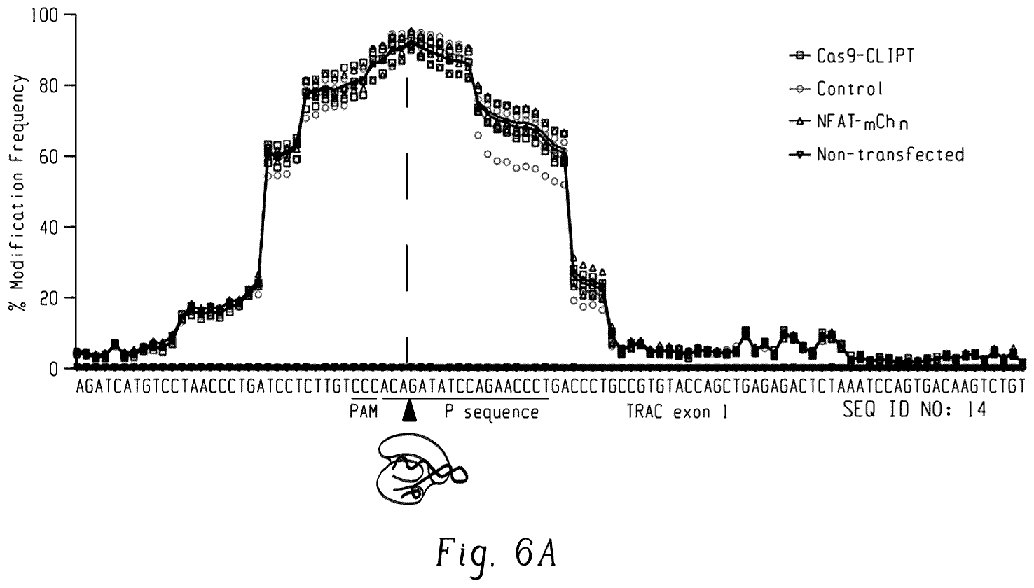

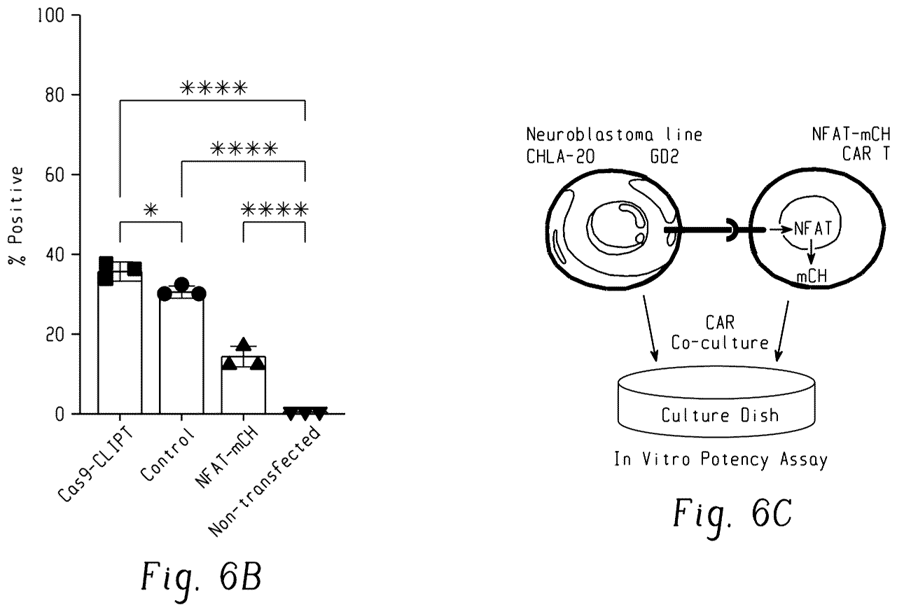

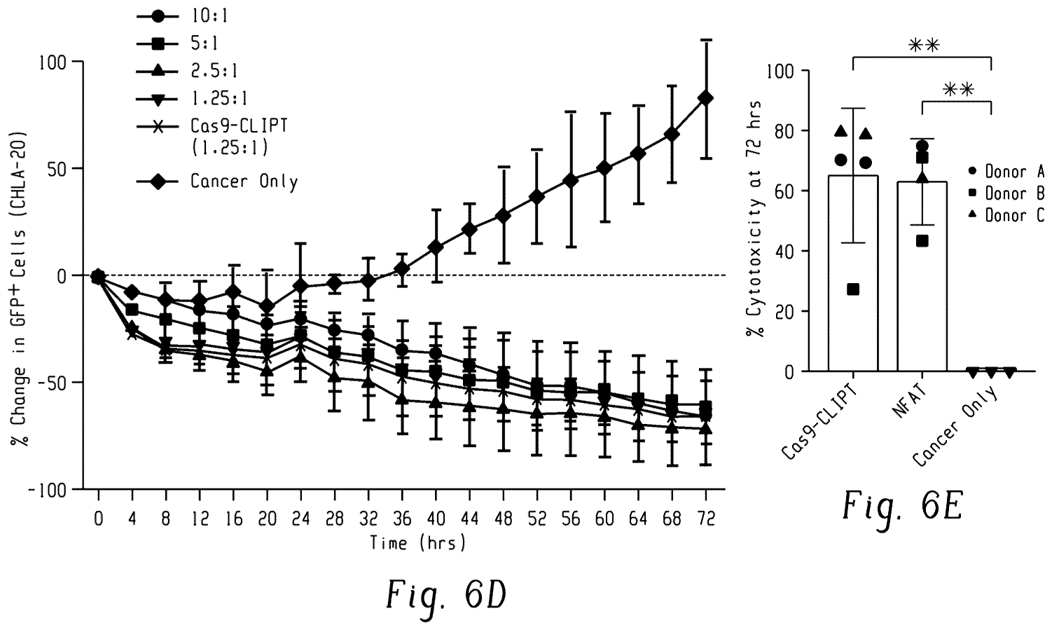

Similarly to the Cas9-CLIPT TRAC-CAR T cells, confirmed precise insertion via HDR of the NFAT-mCh transgene at the genomic level was confirmed via long-read sequencing on genomic DNA isolated from the NFAT-mCh TRAC-CAR T cell products. DNA was extracted on day 10 of manufacturing from NFAT-mCh TRAC-CAR T cells from three different donors. The regions of interest were amplified via PCR around the intended insertion site. Amplicons were sequenced using a long-read Oxford Nanopore instrument. The modification frequency of each base pair around the TRAC cut site relative to the wild-type sequence was calculated for NFAT-mCh TRAC-CAR T cell amplicons by dividing by the number of reads not aligned to the wild-type sequences at each base pair with the total number of reads observed at each base pair. High on-target genome editing was observed, as indicated by high indel formation precisely at the TRAC target site (FIG. 6A). There were no significant differences in the indel pattern between NFAT-mCh, Cas9-CLIPT, and Control TRAC-CAR T cells (FIG. 6A). The allelic knock-in efficiency was estimated by dividing the number of reads aligned to the insert by the total number of reads aligned to both the edited and the unedited samples and precise insertions of NFAT-mCh (NFAT-mCh: 14% (2.4%); p<0.0001) were observed (FIG. 6B).

### Example 8: Potency of NFAT-mCh TRAC-CAR T Cells

Despite the lower expression, NFAT-driven mCherry fluorescence was detected in images of Cas9-CLIPT NFAT-mCh CAR T cells with GD2⁺ neuroblastoma cells on the IncuCyte live-imaging platform (FIG. 6C). mCherry fluorescence was detected for up to 72 hours (data not shown), indicating sustained expression with antigen exposure. The number of mCherry⁺ cells decreased with lower effector: target (E:T) ratios (NFAT-mCh: (10:1) 556 (228), (5:1) 459 (190), (2.5:1) 235 (98), (1.25:1) 141 (49), Cas9-CLIPT: (10:1) 5 (3), (5:1) 2 (1), (2.5:1) 2 (1), (1.25:1) 3 (2); p (10:1, 5:1, 2.5:1, 1.25:1)=0.0004) (Fig. S13) while the average intensity was unchanged at 12 hours (NFAT-mCh: (10:1) 30 (1), (5:1) 29 (5), (2.5:1) 25 (2), (1.25:1) 25 (2)) (data not shown). At 10:1, 5:1, 2.5:1, and 1.25:1 effector: target (E:T) ratios, all groups successfully lysed CHLA-20 cells over 72 hours at each E:T ratio (FIG. 6D) with no differences in the extent of cytotoxicity for a 1.25:1 E:T after 72 hours of co-culture (NFAT-mCh: 64% (14%), Cancer Only 0% (0%); p (NFAT-mCh vs. Cancer Only)=0.005) (FIG. 6E). These results indicate that the Cas9-CLIPT integration strategy can create potent GD2 TRAC-CAR T cells via a functional large transgene.

### Discussion

Demonstrated herein is a streamlined manufacturing process of virus-free TRAC-CAR T cells which simplifies the preparation of donor templates and increases knock-in of small and large transgenes into T cells. By adding a single TRAC target sequence into a CAR-containing Nanoplasmid™, Cas9-CLIPT was synthesized, eliminating the costly, time consuming, and challenging production of linearized HDR donor templates via PCR or restriction digestion of circular plasmids. Nanoplasmid™ DNA can be easily manufactured within recombinant bacteria at scale using good manufacturing practices (GMP) and is already in use in clinical trials, making the Cas9-CLIPT platform adaptable for clinical production of TRAC-CAR T cell therapies. Manufacturing TRAC-CAR T cells with Cas9-CLIPT significantly increases knock-in efficiencies commonly observed when using retroviruses.

CAR knock-in was assisted in some cases by including poly-L-glutamic acid (PGA) during RNP complexing, as the knock-in efficiency decreases without it. PGA is an anionic polymer that is theorized to prevent excess negatively charged unbound guide RNA from associating with the RNP, thus increasing the concentration of active RNP and subsequently the HDR knock-in rate. However, the addition of PGA to other non-viral CAR T cell workflows has had inconsistent effects, with some studies observing seemingly no benefit to knock-in or viability. Electroporation is also influenced by the charge of the cargo, so neutralization of positive charges on Cas9 by PGA may prevent inhibitory interactions with the heterogeneous surface charge on T cells, thereby eliminating a potential barrier to delivery. While we employed PGA, it did not affect the extent of Cas9-CLIPT Nanoplasmid™ linearization, which manifests as a heavier band than linearized Control Nanoplasmid™ (FIG. 1B).

Cas9-CLIPT bound to SpCas9 with nuclear localization signal (NLS) may increase trafficking to the nucleus to increase knock-in, similarly to ssDNA HDR donor templates bound with Cas9 via truncated target sequences. Bound Cas9 may also cleave the donor template and genomic target efficiently, leading to an increased chance of HDR due to the proximity of homologous sequences to the on-target site. Other non-viral processes have incorporated gRNA cut sites to increase the knock-in of a CD19 CAR and a reporter gene. (Webber, B. R. et al. Cas9-induced targeted integration of large DNA payloads in primary human T cells via homology-mediated end-joining DNA repair. Nat Biomed Eng (2023) doi: 10.1038/s41551-023-01157-4). The latter study also integrated a large (6.3 kb) multicistronic CAR transgene, which bypasses the usual 1.5 kb limit for linearized HDR donor templates in T cells. The present study also bypasses this limit by achieving high knock-in with the 5.0 kb inducible NFAT-mCherry GD2-CAR construct with a single gRNA cut site as opposed to a double cut variant. Finally, homology-independent targeted integration, “HITI,” templates with a single cleavage site perform differently from HITI templates with two cleavage sites for non-HDR knock-in within mice in vivo. Mechanisms within mammalian cells that distinguish one versus two cleaved ends of the template may not involve bound Cas9, as with our Cas9-CLIPT strategy, but still promote transgene knock-in for our human T cell system.

Other end joining integration processes like MMEJ or HMEJ are possible with Cas9-CLIPT and could integrate the entire linearized plasmid containing plasmid backbone outside the homology arms. However, in the Cas9-CLIPT design, polyadenylation sequences at the end of the inserted transgene likely prevent the backbone from being transcribed from the endogenous TRAC promoter. MMEJ and HMEJ repair outcomes were not observed, which may be due to Nedisertib addition, known to block end joining. Non-HDR integration events can also be reduced while increasing HDR insertion by simultaneously inhibiting DNA-PK and Polymerase Theta-mediated end joining with small molecule inhibitors, Nedisertib, ART558, and AZD7648. Control TRAC-CAR T cells treated with Nedisertib and ART558 and tripled knock-in compared to untreated cells.

Efficient knock-in on the CEP system indicates the feasibility of moving to automated and scaled-up TRAC-CAR T cell therapy biomanufacturing. This move is likely to have significant benefits compared to the current commercial CAR T cell manufacturing workflow.

The Cas9-CLIPT donor template simplifies non-viral CAR T cell production by eliminating expensive, difficult-to-scale-up dsDNA linearization and purification steps. An 8.8-fold expansion of Cas9-CLIPT TRAC-CAR T cells during manufacturing while increasing knock-in to over 60% with a 2.2 kb insert was demonstrated. Compared to using rAAV donor templates, an inducible fluorescent reporter (5.5 kb transgene) at rates exceeding 35% could be inserted by bypassing the cargo limit of 4.5 kb for rAAV. Cas9-CLIPT likely has the potential to knock-in even larger transgenes, enabling the inclusion of co-expression of additional genes.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A DNA template plasmid for generating genome modified immune cells, comprising a plasmid backbone, a first insert, and a second insert;
wherein the first insert comprises a transgene, wherein the transgene is flanked by left and right homology arms that are complementary to sequences on both sides of a cleavage site in a target expressed gene in an unmodified immune cell;
wherein the second insert comprises a cleavage target comprising a protospacer sequence defining the cleavage site in the target expressed gene, and a protoadjacent motif sequence (PAM) for recognition by a Cas9 ribonucleoprotein complex (Cas9-RNP);
wherein the Cas9-RNP comprises a Cas9 polypeptide and a single guide RNA (sgRNA) comprising a sequence complementary to the protospacer sequence; and
wherein the Cas9-RNP binds the second insert and linearizes the DNA template plasmid by Cas9-RNP-directed cleavage at the cleavage site.

2. The DNA template plasmid of claim 1, wherein the plasmid backbone comprises 500 base pairs or less and does not include an antibiotic-resistance gene.

3. The DNA template plasmid of claim 2, wherein the plasmid backbone comprises a plasmid having RNA-OUT antibiotic-free selection and an R6K origin of replication, or a plasmid comprising an R6K origin of replication and no selection marker.

4. The DNA template plasmid of claim 1, wherein the right and left homology arms are each independently 50 to 3000 nucleotides in length.

5. The DNA template plasmid of claim 1, wherein first insert comprises a splice acceptor site (SA), encodes a self-cleaving peptide (2A), includes a terminator sequence that defines the end of a transcriptional sequence (polyA), or a combination thereof.

6. The DNA template plasmid of claim 5, wherein 2A comprises a coding sequence for a porcine teschovirus-1 (P2A) peptide, a Thoseasigna virus (T2A) peptide, an equine rhinitis A virus (E2A) peptide, or a foot-and-mouth disease virus (F2A) peptide.

7. The DNA template plasmid of claim 1, wherein the transgene of the first insert comprises a chimeric antigen receptor (CAR).

8. The DNA template plasmid of claim 7, wherein the CAR comprises an extracellular domain linked to an intracellular domain through a first transmembrane domain, wherein the first extracellular domain comprises an antigen recognition domain.

9. The DNA template plasmid of claim 8, wherein the antigen recognition domain binds an autoimmune or inflammatory disease antigen, a cardiac disease antigen, a senescence disease associated antigen, a disease associated antigen, or a tumor-specific antigen.

10. The DNA template plasmid of claim 9, wherein the tumor-specific antigen comprises carbonic anhydrase IX (CAIX), carcinoembryonic antigen (CEA), CD5, CD8, CD7, CD10, CD19, CD20, CD22, CD30, CD33, CLL1, CD34, CD38, CD41, CD44, CD49f, CD56, CD74, CD133, CD138, CD123, CD44V6, Claudin-18, B7 homolog 3 protein (B7-H3), fibroblast activation protein (FAP), cancer antigen 19 (CA19), an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinases erb-B2,3,4 (erb-B2,3,4), folate-binding protein (FBP), fetal acetylcholine receptor (AChR), adult AChR subunits, folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), K-light chain, kinase insert domain receptor (KDR), Lewis Y (LeY), L1 cell adhesion molecule (LICAM), melanoma antigen family A, 1 (MAGE-A1), Mucin 16 (MUC16), Mucin 1 (MUC1), Mesothelin (MSLN), PSMA, GPC3, ERBB2, MAGEA3, p53, MART1, GP100, Proteinase3 (PR1), Tyrosinase, Survivin, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGFR, EGFR-vIII, CD99, CD70, ADGRE2, CCR1, LILRB2, PRAME CCR4, CD5, CD3, TRBC1, TRBC2, TIM-3, Integrin B7, ICAM-1, CD70, Tim3, CLEC12A, ERBB, or a combination thereof.

11. The DNA template plasmid of claim 8, wherein the transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof, or wherein the single transmembrane domain comprises CD4, CD8, CD-8 alpha, CD8-beta, CD3-epsilon, CD3-beta, CD28, 4-1BB, OC40, PD-1, LAG-3, CH2CH3 or NKG2D, IgG, CD3-ζ, or a combination thereof.

12. The DNA template plasmid of claim 8, wherein the intracellular domain comprises a costimulatory domain selected from 2B4, CD27, CD28, CD137, CD154, CD244, CD278, and combinations thereof, and a signaling domain selected from CD16, DAP10, DAP12, CD28, ICOS, CD27, OX40, CD40L, CD3-ζ, and combinations thereof.

13. An RNP complex comprising the DNA template plasmid of claim 1, a Cas9 polypeptide, and an sgRNA.

14. An ex vivo, virus-free method of site-specifically inserting a transgene into an immune cell expressed gene to generate a genome modified immune cell, comprising
providing the DNA template plasmid of claim 1;
incubating the DNA template plasmid with the Cas9-RNP for a time sufficient to linearize the DNA template plasmid by Cas9-RNP-directed cleavage at the cleavage site; and
introducing into a population of unmodified immune cells the Cas9-RNP and linearized DNA template plasmid to provide the genome modified immune cells, wherein, in the genome modified immune cells, the transgene is specifically integrated into the cleavage site of the immune cell expressed gene.

15. The method of claim 14, wherein the immune cell is a T-cell, a Natural Killer (NK) cell, an innate lymphoid cell, a Cytokine Induced Killer (CIK) cell, a hematopoietic progenitor cell, a peripheral blood (PB) derived immune cell, a bone marrow derived immune cell, a macrophage, or an umbilical cord blood (UCB) derived immune cell.

16. The method of claim 14, wherein introducing the Cas9-RNP and linearized DNA template plasmid comprises introducing Cas9-RNP bound to the linearized DNA template plasmid and unbound Cas9-RNP.

17. The method of claim 14, further comprising administering the genome modified immune cell to a subject in need of such treatment.

18. The method of claim 17, wherein the subject is in need of treatment for a solid tumor selected from a sarcoma, adrenocortical carcinoma, retinoblastoma, kidney cancer, bladder cancer, breast cancer, neuroblastoma, melanoma, sarcoma, neuroendocrine cancer, colorectal cancer, lung cancer, head and neck cancer, prostate cancer, pancreatic cancer, ovarian cancer, uterine cancer, oral cavity cancer, glioblastoma, lymphoma, diffuse midline glioma, carcinoid tumors, neuroendocrine tumors, thyroid cancer, liver cancer, or a combination thereof.

19. The method of claim 17, wherein the subject is in need of treatment for a hematologic malignancy.

20. The method of claim 17, wherein the subject is in need of treatment for a neurodegenerative disease, stroke, craniocerebral trauma and/or accident, or an elderly patient in need of treatment for aging.
